(19) United States
(12) Patent Application Publication
(10) Pub. No.: US 2026/0199293 A1
(43) Pub. Date: Jul. 16, 2026

(54) 2-Arylbenzimidazole Compounds for the Treatment of Hemoglobinopathies

(71) Applicant: TRANQUIS THERAPEUTICS, INC., Palo Alto, CA (US)

(72) Inventors: Sanjay Kumar Kakkar, London (GB); Jonas O'Gara Hannestad, San Mateo, CA (US)

(21) Appl. No.: 19/135,040

(22) PCT Filed: Nov. 30, 2023
(86) PCT No.: PCT/US2023/081849

§ 371 Date: Jun. 2, 2025

Related U.S. Application Data
(60) Provisional application No. 63/385,859, filed on Dec. 2, 2022.

(51) Int. Cl. A61K 31/4184; A61K 31/17; A61K 31/198; A61K 31/427; A61K 31/437; A61K 31/4439; A61K 31/444; A61K 31/519; A61P 7/06; C07K 16/28

(52) U.S. Cl. CPC A61K 31/4184; A61K 31/17; A61K 31/198; A61K 31/427; A61K 31/437; A61K 31/4439; A61K 31/444; A61K 31/519; A61P 7/06; C07K 16/2854

(57) ABSTRACT

The present disclosure provides 2-arylbenzimidazole compounds and pharmaceutical compositions or formulations thereof for the treatment of hemoglobinopathies in a subject. In some embodiments, the hemoglobinopathy is a sickle cell disease (SCD). The present disclosure also provides 2-arylbenzimidazole compounds and pharmaceutical compositions or formulations thereof for increasing fetal hemoglobin (HbF) production in a subject.

### 1. BACKGROUND

Hemoglobinopathies encompass a number of anemias of genetic origin in which there is a decreased production of red blood cells (RBCs) and/or increased destruction (hemolysis) of RBCs. Hemoglobinopathies also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent hemoglobin A (HbA). Sickle cell disease results from a point mutation in the β-globin structural gene, leading to the production of an abnormal (sickled) hemoglobin (HbS). HbS RBCs are more fragile than normal RBCs and undergo hemolysis more readily, leading eventually to anemia. Sickling of RBCs in blood vessels also leads to impairment in blood flow (vaso-occlusion), with resulting ischemia, endothelial injury, and sterile inflammation in multiple organs.

More specifically, sickle cell disease (SCD)—also known as sickle-cell anemia (SCA, which more precisely is used to identify the most common genotypes that causes SCD, homozygosity for HbSS)—is characterized by RBCs that adopt an abnormal, rigid, sickle shape, referred to as “sickling” under low-oxygen conditions. The abnormal cells with the potential to sickle are referred to as sickle red blood cells (SRBC). Repeated episodes of sickling can damage the blood cell's membrane and decrease its deformability. Some of the sickled cells can fail to return to normal shape when normal oxygen tension is restored. These irreversibly sickled SRBC and reversibly sickled SRBC are rigid and unable to deform as they pass through narrow capillaries, leading to vessel occlusion and ischemia. The actual anemia of the illness is caused mainly by hemolysis, the premature destruction of the SRBC.

Improved compounds and formulations for the treatment of sickle cell disease are needed.

The compound (2-(4-tert-butylphenyl)-1H-benzimidazole), known alternatively as ZLN-005 and as TQS-168, is known to be an activator of Ppargc1α (PGC-1α) expression (Zhang et al., Diabetes 62:1297-1307 (2013)). Recently, upregulation of PGC-1α by ZLN-005 in human primary erythroid progenitor CD34+ cells was shown to induce both fetal γ-globin mRNA and protein expression as well as the percentage of HbF-positive cell (F cells), without significantly affecting cell proliferation and differentiation. Sun et al., Br. J. Haematol. 197:97-109 (2022). The authors further found that the combination of ZLN005 and hydroxyurea (hydroxycarbamide) exhibited an additive effect on the expression of γ-globin and the generation of F cells from cultured CD34+ cells, and that ZLN005 induced robust expression of the murine embryonic βh1-globin gene, and to a lesser extent, human γ-globin gene expression, in sickle mice. Hydroxyurea is an approved treatment for SCD that reduces the incidence of vaso-occlusive crises by increasing the amount of HbF in RBCs. RBCs with higher levels of HbF are less prone to sickling. Dosing with hydroxyurea is often limited by tolerability; therefore, an additional oral medication that can increase HbF levels that could be taken concurrently with hydroxyurea could enable patients with SCD to further increase HbF levels in RBCs and thus further reduce the incidence and severity of vaso-occlusive crises and other complications of SCD.

Oral administration is preferred to parenteral administration for chronic life-long treatment, as is required for treating hemoglobinopathies such as sickle cell disease. ZLN-005/TQS-168 is poorly soluble, making formulation for oral administration difficult. There is a need, therefore, for agents that increase PGC-1α expression that can be administered orally for treatment of hemoglobinopathies such as SCD.

### 2. SUMMARY

The present disclosure provides 2-arylbenzimidazole compounds and pharmaceutical compositions or formulations for the treatment of hemoglobinopathies in a subject. In some embodiments, the hemoglobinopathy is a sickle cell disease (SCD). The present disclosure also provides 2-arylbenzimidazole compounds and pharmaceutical compositions or formulations for increasing fetal hemoglobin (HbF) production in a subject.

As summarized above, the present disclosure provides 2-arylbenzimidazole compounds (e.g., of formula (I), as described herein) and improved formulations of TQS-168 (e.g., comprising amorphous solid dispersions as described herein) for the treatment of hemoglobinopathies in a subject. Also provided herein are 2-arylbenzimidazole compounds (e.g., of formula (I), as described herein), and improved formulations of TQS-168 (e.g., comprising amorphous solid dispersions as described herein) for use in increasing fetal hemoglobin (HbF) production in a subject.

The methods of treating hemoglobinopathies and increasing fetal hemoglobin (HbF) production with 2-arylbenzimidazole compounds and improved formulations of TQS-168 are described in greater detail below.

TQS-168 (2-(4-tert-butylphenyl)-1H-benzimidazole), previously known as ZLN-005, is known to be an activator of Ppargc1α (PGC-1α) expression (Zhang et al., Diabetes 62:1297-1307 (2013)).

TQS-168 has previously been shown to suppress myeloid-mediated inflammation and reduce disease severity in murine models of neurodegenerative diseases in which neuroinflammation contributes to the underlying pathophysiology, including Parkinson's disease, Alzheimer's disease, and amyotrophic lateral sclerosis (ALS) (U.S. Pat. Nos. 10,272,070; 10,583,125; and 10,653,669, the disclosures of which are incorporated herein by reference in their entireties). TQS-168 has also been shown to suppress metabolic dysfunction in microglia in older mice, inhibit inflammatory cytokine production in microglia in older mice, suppress systemic inflammation in older mice, and alleviate behavioral dysfunction in older mice (U.S. Pat. No. 10,653,669, the disclosure of which is incorporated herein by reference in its entirety). TQS-168 and its analogs have also been shown to suppress acute systemic immune activation, including cytokine release syndrome (CRS) (WO2021/262617, the disclosure of which is incorporated herein by reference in its entirety). TQS-168 has also been shown to induce γ-globin expression and fetal hemoglobin (HbF) production in sickle mice (Sun et al., Br J Haematol. 197:97-109 (2022)). The effects on both myeloid cells and erythroid cells appear to occur through the increase of PGC-1α gene expression.

As disclosed herein a range of 2-arylbenzimidazole compounds (e.g., of formula (I), as described herein) can be administered to treat hemoglobinopathies, such as a sickle cell disease. Also disclosed herein are improved formulations of 2-(4-tert-butylphenyl)-1H-benzimidazole (TQS-168) (e.g., comprising amorphous solid dispersions as described herein) that can be administered to treat hemoglobinopathies, such as a sickle cell disease. Also provided herein are 2-arylbenzimidazole compounds (e.g., of formula (I), as described herein), and improved formulations of TQS-168 (e.g., comprising amorphous solid dispersions as described herein) for use in increasing fetal hemoglobin (HbF) production in a subject.

Accordingly, in a first aspect, there is provided a method of treating a hemoglobinopathy in a subject, comprising administering to a subject suffering from a hemoglobinopathy an effective amount of a compound of formula (I):

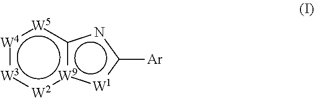

or a pharmaceutically acceptable salt, solvate, deuterated derivative, or prodrug wherein:
Ar is

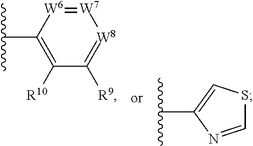

W¹ is chosen from O, S, and N—R¹, or, when W⁹ is N, W¹ may additionally be C—R⁵⁰;
W² is N or C—R²;
W³ is N or C—R³;
W⁴ is N or C—R⁴;
W⁵ is N or C—R⁵;
W⁶ is N or C—R⁶;
W⁷ is N or C—R⁷;
W⁸ is N or C—R⁸;
W⁹ is C, or, when W¹ is C—R⁵⁰; W⁹ may be N;
wherein:
R¹ is chosen from H, (C₁-C₃)alkyl, —CH₂OC(═O)R³⁰; —CH₂OP(═O)OR⁴⁰OR⁴¹, —C(═O)OR⁴², and —C(═O)R⁴³;
wherein:
R³⁰ is chosen from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴NHR⁴⁵, and guanidino;
wherein:
R⁴⁴ is chosen from any naturally occurring amino acid sidechain;
R⁴⁵ is chosen from H, methyl, and (C₁-C₄)alkoxycarbonyl;
R⁴⁰ and R⁴¹ are chosen independently from hydrogen and (C₁-C₆)hydrocarbyl;
R⁴² is (C₁-C₅)alkyl;
R⁴³ is (C₁-C₃)alkyl; and
R⁵⁰ is H or (C₁-C₃)alkyl;
R², R³, R⁴ and R⁵ are chosen independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are chosen independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are chosen independently from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy,

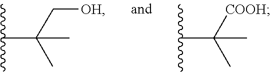

and
R⁸ is chosen from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino,

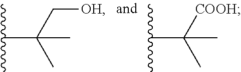

with the proviso that:
i) when Ar is

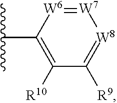

R⁸ is not (C₁-C₄)alkyl when:
(a) W¹ is N—R¹;
(b) R¹ is hydrogen;
(c) W², W³, W⁴, W₅, W⁶, and W⁷ are C—H;
(d) W⁸ is C-RA;
(e) W⁹ is C; and
(f) R⁹ and R¹⁰ are hydrogen.

In another aspect, there is provided a method of increasing fetal hemoglobin (HbF) production, the method comprising administering to a subject in need of increased HbF production an effective amount of a compound of formula (I) (e.g., as described herein), or a pharmaceutically acceptable salt, a solvate, or prodrug thereof.

In another aspect, there is provided a method of treating a hemoglobinopathy, comprising administering to a subject suffering from a hemoglobinopathy an effective amount of a pharmaceutical composition comprising:
an amorphous solid dispersion of 2-(4-tert-butylphenyl)-1H-benzimidazole (TQS-168) or a pharmaceutically acceptable salt thereof; and
a pharmaceutically acceptable carrier matrix.

In yet another aspect, there is provided a method of increasing fetal hemoglobin (HbF) production, the method comprising administering to a subject in need of increased HbF production an effective amount of a pharmaceutical composition comprising:
an amorphous solid dispersion of 2-(4-tert-butylphenyl)-1H-benzimidazole (TQS-168) or a pharmaceutically acceptable salt thereof; and
a pharmaceutically acceptable carrier matrix.

In some embodiments of the subject methods, the hemoglobinopathy is a sickle cell disease. In some embodiments, the sickle cell disease is sickle cell anemia (HbSS).

### 3. DETAILED DESCRIPTION

### 3.1. Methods of Use

In one aspect, methods are provided for treating a hemoglobinopathy. The methods comprise administering to a subject suffering from a hemoglobinopathy an effective amount of a compound of formula (I), as described hereinbelow, or a pharmaceutically acceptable salt, solvate, deuterated derivative, or prodrug thereof.

In another aspect, further methods are provided for treating a hemoglobinopathy. The methods comprise administering to a subject suffering from a hemoglobinopathy an effective amount of a pharmaceutical composition comprising an amorphous solid dispersion of (2-(4-tert-butylphenyl)-1H-benzimidazole) (TQS-168), or a pharmaceutically acceptable salt, solvate, deuterated derivative, or prodrug thereof.

In another aspect, methods are provided for increasing HbF production. The methods comprise administering to a subject in need of increased HbF production an effective amount of a compound of formula (I), as described hereinbelow, or a pharmaceutically acceptable salt, solvate, deuterated derivative, or prodrug thereof.

In another aspect, methods are provided for increasing HbF production. The methods comprise administering to a subject in need of increased HbF production an effective amount of a pharmaceutical composition comprising an amorphous solid dispersion of (2-(4-tert-butylphenyl)-1H-benzimidazole) (TQS-168), or a pharmaceutically acceptable salt, solvate, deuterated derivative, or prodrug thereof.

### 3.1.1. Methods of Treating Hemoglobinopathies

In some embodiments of the methods for treating a hemoglobinopathy, the subject has been diagnosed with a hemoglobinopathy. In some embodiments, the patient has been clinically diagnosed with a hemoglobinopathy based on symptoms. In some embodiments, the patient has been diagnosed based on genotype. In some embodiments, the patient has been diagnosed based on symptoms and on genotype.

In some embodiment of the methods described herein, the hemoglobinopathy is a β-hemoglobinopathy. In some embodiments, the hemoglobinopathy is a sickle cell disease (SCD), a sickle cell anemia, a sickle cell trait (SCT), or a β-thalassemia. In some embodiments, the hemoglobinopathy is a β-thalassemia. In some embodiments, the hemoglobinopathy is a sickle cell disease (SCD). In some embodiments, the hemoglobinopathy is a sickle cell trait (SCT).

In some embodiments, the SCD is selected from sickle cell anemia (HbSS), sickle-hemoglobin C disease (HbSC), sickle beta-plus-thalassemia (HbS/β+), and sickle beta-zero-thalassemia (HbS/βO).

In some embodiments, the SCD is sickle cell anemia (HbSS).

In some embodiments, the SCD is sickle-hemoglobin C disease (HbSC).

In some embodiments, the SCD is sickle beta-plus-thalassemia (HbS/β+).

In some embodiments, the SCD is sickle beta-zero-thalassemia (HbS/βO).

In some embodiments of the methods described herein, the subject is identified as suffering from a sickle cell crisis, or has been identified as being at risk of incurring a sickle cell crisis prior to treatment.

The treatment according to the present method can ameliorate one or more symptoms associated with the hemoglobinopathy, such as anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris, by increasing the amount of fetal hemoglobin in the individual

In some embodiments, the method further comprises administering to the subject one or more additional active agents, including additional active agents described herein.

### 3.1.2. Methods of Increasing Fetal Hemoglobin (HbF) Production

In some embodiments of the methods for increasing HbF production, the subject in need of increased HbF production has a hemoglobinopathy, including the hemoglobinopathies described in Section 3.1.1 herein. In some embodiments, the subject in need of increased HbF production has sickle cell anemia. In some embodiments, the subject in need of increased HbF production is heterozygous for sickle cell trait.

In various embodiments, the percentage of HbF production in the subject is increased by 1% or more, such as 2% or more, 3% or more, 4% or more, 5% or more, 6% or more, 7% or more, 8% or more, 9% or more, 10% or more, or even more, relative to a mean baseline level of HbF in RBCs in the subject before treatment according to the methods described herein. In some embodiments of the methods described herein, the percentage of HbF production in the subject is increased by 10% or more, such as 20% or more, 30% or more, 40% or more, 50% or more, 60% or more, 70% or more, 80% or more, 90% or more, relative to a baseline level of HbF production in the subject before treatment according to the methods described herein. In some embodiments, increasing HbF production in a subject means increasing the HbF production by a factor of 2 or more, such as 3 or more, 4 or more, 5 or more, 10 or more, 100 or more, or even more, relative to a baseline level of HbF production in the subject before treatment according to the methods described herein.

In some embodiments, the method further comprises administering to the subject one or more additional active agents, including those described herein below.

### 3.1.3. Subject Age

In typical embodiments, the subject is a human patient.

In some embodiments, the human patient is under the age of 1 year. In some embodiments, the human patient is 2-5 years old, 5-10 years old, 10-15 years old, 15-20 years old, 20-25 years old, 25-30 years old, 30-35 years old, or 35-40 years of age.

In some embodiments, the human is no more than 40 years of age. In some embodiments, the human is no more than 5 years of age. In some embodiments, the human is no more than 10 years of age. In some embodiments, the human is no more than 15 years of age. In some embodiments, the human is no more than 20 years of age. In some embodiments, the human is no more than 25 years of age. In some embodiments, the human is no more than 30 years of age. In some embodiments, the human is no more than 40 years of age, such as no more than 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 years of age, or no more than 1 year of age.

In some embodiments, the human is over 40 years of age. In some embodiments, the human is over 45 years of age. In some embodiments, the human is over 50 years of age. In some embodiments, the human is over 55 years of age. In some embodiments, the human is over 60 years of age. In some embodiments, the human is over 65 years of age. In some embodiments, the human is over 70 years of age. In some embodiments, the human is over 75 years of age. In some embodiments, the human is over 80 years of age. In some embodiments, the human is over 85 years of age. In some embodiments, the human is over 90 years of age.

In various embodiments, the human is 30 to 100 years of age, such as 30 to 40 years, 30 to 50 years, 30 to 60 years, 30 to 70 years, 30 to 80 years, 30 to 90 years, 40 to 50 years, 40 to 60 years, 40 to 70 years, 40 to 80 years, 40 to 90 years, 40 to 100 years, 50 to 60 years, 50 to 70 years, 50 to 80 years, 50 to 90 years, 50 to 100 years, 60 to 70 years, 60 to 80 years, 60 to 90 years, 60 to 100 years, 70 to 80 years, 70 to 90 years, 70 to 100 years, 80 to 90 years, 80 to 100 years, or 90 to 100 years of age.

### 3.2. 2-Arylbenzimidazole Compounds

In certain aspects, the methods of treating a hemoglobinopathy and/or for increasing production of HbF in a subject comprise administering a therapeutically effective amount of one or more compounds of formula (I), or a pharmaceutically acceptable salt, solvate, deuterated derivative, or prodrug thereof to the subject in need thereof, where Formula (I) is

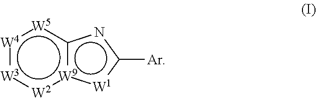

In some embodiments of formula (I), the compound is of formula (II):

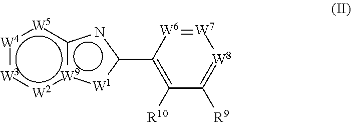

wherein:
W² is N or C—R²
W³ is N or C—R³;
W⁴ is N or C—R⁴;
W⁵ is N or C—R⁵;
W⁶ is N or C—R₆;
W⁷ is N or C—R⁷;
W⁸ is N or C—R⁸;
wherein:
R¹ is selected from —CH₂OC(═O)R³⁰, —CH₂OP(═O)OR⁴⁰OR⁴¹, C(═O)OR⁴², and C(═O)R⁴³;
   wherein:
R³⁰ is selected from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
   wherein:
   R⁴⁴ is selected from any naturally occurring amino acid sidechain; and
   R⁴⁵ is selected from H, methyl, and (C₁-C₄)alkoxycarbonyl; and
   R⁴⁰ and R⁴¹ are selected independently from hydrogen and (C₁-C₆)hydrocarbyl;
   R⁴² is (C₁-C₅)alkyl; and
   R⁴³ is (C₁-C₃)alkyl; and
   R², R³, R⁴ and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
   R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
   R⁷ and R⁹ are selected independently from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
   R⁸ is chosen from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino; with the proviso that R⁸ is not hydrogen or (C₁-C₄)alkyl when:
(a) W¹ is N—R¹;
(b) R¹ is hydrogen;
(c) W², W³, W⁴, W⁵, W⁶, and W⁷ are C—H;
(d) W⁸ is C—R⁸;
(e) W⁹ is C; and
(f) R⁹ and Ri^{o} are hydrogen.

In some embodiments of the compound of formula (II):
(a) W¹ is N—R¹ and R¹ is hydrogen;
(b) W² is C—R², wherein R² is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(c) W³ is C—R³, wherein R³ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(d) W⁴ is C—R⁴, wherein R⁴ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(e) W⁵ is C—R⁵, wherein R⁵ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino [—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(f) W⁶ is C—R⁶, wherein R⁶ is selected from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
(g) W⁷ is C—R⁷, wherein R⁷ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy;
(h) W⁸ is C—R⁸, wherein R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino;
(i) W¹ is NR¹, O, or S and W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is selected from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino.

In some embodiments of the compound of formula (II):
(a) W¹ is N—R¹ and R¹ is hydrogen;
(b) W² is C—R², wherein R² is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(c) W³ is C—R³, wherein R³ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(d) W⁴ is C—R⁴, wherein R⁴ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(e) W⁵ is C—R⁵, wherein R⁵ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(f) W⁶ is C—H;
(g) W⁷ is C—R⁷, wherein R⁷ is hydrogen or (C₃-C₄)alkyl;
(h) W⁸ is C—R⁸, wherein R⁸ is selected from H, (C₁-C₄)alkyl, amino, (C₁-C₄)alkoxy, halo(C₁-C₄)alkoxy, and hydroxy;
(i) W¹ is NR¹; and W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is hydrogen.

In some embodiments of the compound of formula (II):
(a) W¹ is N—R¹ and R¹ is hydrogen;
(b) W² is selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
(c) W³ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br;
(d) W⁴ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br;
(e) W⁵ is selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
(f) W⁶ is C—H;
(g) W⁷ is C—R⁷, wherein R⁷ is hydrogen or (C₃-C₄)alkyl;
(h) W⁸ is C—R⁸, wherein R⁸ is selected from H, tert-butyl, amino, and methoxy;
(i) W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is hydrogen.

In some embodiments, of formula (I) or (II), the compound is of formula (III):

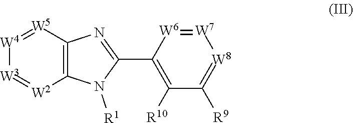

wherein:
R¹ is selected from —CH₂OC(═O)R³⁰, —CH₂OP(═O)OR⁴⁰OR⁴¹, —C(═O)OR⁴², and —C(═O)R⁴³; where R³⁰ is selected from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino; where R⁴⁴ is chosen from any naturally occurring amino acid sidechain; R⁴⁵ is chosen from H, methyl, and (C₁-C₄)alkoxycarbonyl; R⁴⁰ and R⁴¹ are chosen independently from hydrogen and (C₁-C₆)hydrocarbyl; R⁴² is (C₁-C₅)alkyl; and R⁴³ is (C₁-C₃)alkyl;
W² is N or C—R²;
W³ is N or C—R³;
W⁴ is N or C—R⁴;
W⁵ is N or C—R⁵;
W⁶ is N or C—R⁶;
W⁷ is N or C—R⁷;
W⁸ is N or C—R⁸;
R², R³, R⁴ and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino.

In some embodiments of formula (III):
W⁶ is C—H; W⁸ is C—R⁸;
R⁹ and R¹⁰ are hydrogen;
R⁷ is H or (C₃-C₄)alkyl;
R⁸ is selected from H, (C₁-C₄)alkyl, amino, (C₁-C₄)alkoxy, halo(C₁-C₄)alkoxy, and hydroxy;
W² and W⁵ are C—R² and C—R⁵, respectively;
R², R³, R⁴, and R⁵ are independently selected from H, halo, and perfluoro(C₁-C₃)alkyl; and
R¹ is —CH₂OP(═O)OR⁴⁰OR⁴¹ or —CH₂OC(═O)R³⁰.

In some embodiments of formula (III):
W² and W⁵ are independently selected from C—H, C—F, C-D, C—CF₂H, C-CD₃, and C—CF₃;
W³ is selected from N, C—H, C—NH₂, C—F, C—CF₃, C—CF₂H, C-CD₃, and C-D;
W⁴ is selected from N, C—H, C—NH₂, C—F, C—CF₃, C—CF₂H, C-CD₃, and C-D;
W⁷ is C—H;
R^{s} is selected from H, tert-butyl, amino, and methoxy; and
R⁹ is H or tert-butyl.

In some embodiments of formula (III), from one to four of W², W³, W⁴ and W⁵ are C-D or C—F. In some embodiments, one of W², W³, W⁴ and W⁵ is C-D or C—F. In some embodiments, two of W², W³, W⁴ and W⁵ are C-D or C—F. In some embodiments, three of W², W³, W⁴ and W⁵ are C-D or C—F. In some embodiments, all of W², W³, W⁴ and W⁵ are C-D or C—F.

In some embodiments of formula (III), W², W³, W⁴ and W⁵ are C—R², C—R³, C—R⁴ and C—R⁵ respectively, and one of R², R³, R⁴ and R⁵ is perfluoro(C₁-C₄)alkyl, C₁-C₄)alkyl, (C₁-C₄)alkoxy, perfluoro(C₁-C₄)alkoxy, amino, hydroxy, nitrile, halo or carboxamide, and the remainder are hydrogen.

In some embodiments of formula (III), wherein one of W³ or W⁴ is N and the other, along with W² and W⁵, is C—H.

In some embodiments of formula (III), W², W³, W⁴, W⁵, W⁶, and W⁷ are C—H.

In some embodiments of formula (III), R⁸ is t-butyl.

In some embodiments, the compound of formula (I) is of the formulae (IVA)-(IVE):

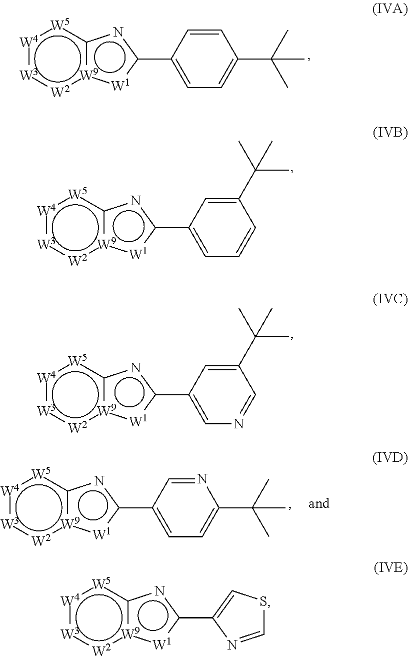

wherein:
W¹ is selected from O, S, and N—R¹, or, when W⁹ is N, W¹ may additionally be C—R⁵⁰;
W² is N or C—R²
W³ is N or C—R³;
W⁴ is N or C—R⁴;
W⁵ is N or C—R⁵;
W⁹ is C, or, when W¹ is C—R⁵⁰; W⁹ may be N;
wherein:
R¹ is selected from H, (C₁-C₃)alkyl —CH₂OC(═O)R³⁰; CH₂OP(═O)OR⁴⁰OR⁴¹, —C(═O)OR⁴², and —C(═O)R⁴³;
wherein:
R³⁰ is selected from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
wherein:
R⁴⁴ is selected from any naturally occurring amino acid sidechain;
R⁴⁵ is selected from H, methyl, and (C₁-C₄)alkoxycarbonyl;
R⁴⁰ and R⁴¹ are selected independently from hydrogen and (C₁-C₆)hydrocarbyl;
R⁴² is (C₁-C₅)alkyl;
R⁴³ is (C₁-C₃)alkyl;
R⁵⁰ is H or (C₁-C₃)alkyl; and
R², R³, R⁴ and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
with the proviso that the compound is of formula (IVA) only when:
W⁹ is C;
W¹ is N—R¹; and
R¹ is not H or (C₁-C₃)alkyl.

In some embodiments of the compound of any one of formulae (IVA)-(IVE):
W² and W⁵ are C—R² and C—R⁵, respectively; and
R², R³, R⁴, and R⁵ are independently selected from H, deuterium, halo, (C₁-C₃)alkoxy, perfluoro(C₁-C₃)alkoxy, nitrile, amino, hydroxyl, aminocarbonyl, (C₁-C₃)alkyl, and perfluoro(C₁-C₃)alkyl.

In some embodiments of the compound of any one of formulae (IVA)-(IVE):
W¹ is N—H or C—H;
W² and W⁵ are independently selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
W³ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br; and
W⁴ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br.

In some embodiments of the compound of any one of formulae (IVA)-(IVE):
W² and W⁵ are independently selected from C—H, C—F, C-D, C—CF₂H, C-CD₃, and C—CF₃;
W³ is selected from N, C—H, C—NH₂, C—F, C—CF₃, C—CF₂H, C-CD₃, and C-D; and
W⁴ is selected from N, C—H, C—NH₂, C—F, C—CF₃, C—CF₂H, C-CD₃, and C-D.

In some embodiments of the compound of any one of formulae (IVA)-(IVE):
from one to four of W², W³, W⁴ and W⁵ are C-D or C—F.

In some embodiments of the compound of any one of formulae (IVA)-(IVE):
one of R², R³, R⁴ and R⁵ is perfluoro(C₁-C₄)alkyl, (C₁-C₄)alkyl, (C₁-C₄)alkoxy, perfluoro(C₁-C₄)alkoxy, amino, hydroxy, nitrile, halo or carboxamide, and the remainder are hydrogen.

In some embodiments of the compound of any one of formulae (IVA)-(IVE):
one of W³ or W⁴ is N and the other, along with W² and W⁵, is C—H.

In some embodiments of the compound of any one of formulae (IVA)-(IVE):
W², W³, W⁴, and W⁵ are C—H.

In the embodiments described below, the compound may be of formula (I), (II), (III), or (IVA)-(IVE) unless otherwise indicated.

In some embodiments of formulae (I), (II) and (IVA)-(IVE), W¹ is N—R¹. In other embodiments of formulae (I), (II) and (IVA)-(IVE), W¹ is O. In yet other embodiments of formulae (I), (II) and (IVA)-(IVE), W¹ is S. In still other embodiments of formulae (I), (II) and (IVA)-(IVE), W¹ is C—R⁵⁰, such as when W⁹ is N. In some embodiments where W¹ is C—R⁵⁰, R⁵⁰ is H. In other embodiments where W¹ is C—R⁵⁰, R⁵⁰ is (C₁-C₃)alkyl.

In some embodiments of formulae (I), (II), (III), and (IVA)-(IVE), R¹ is —CH₂OC(═O)R³⁰; wherein R³⁰ is chosen from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, and guanidino.

In some embodiments when R¹ is —CH₂OC(═O)R³⁰, R³⁰ is chosen from: (a) (C₁-C₆)alkyl; (b) phenyl substituted with (C₁-C₄)alkylamino; (c) the descarboxy residue of a natural amino acid; (d) (C₁-C₃)hydrocarbyl substituted with carboxyl; (e) (C₁-C₅)oxaalkyl; and (d) pyridyl.

In some embodiments of formulae (I), (II) and (IVB)-(IVE), R¹ is H. In other embodiments of formulae (I), (II) and (IVB)-(IVE), R¹ is (C₁-C₃)alkyl.

In some embodiments of formulae (I), (II), (III), and (IVA)-(IVE), W² is N. In other embodiments of formulae (I), (II), (III), and (IVA)-(IVE) W² is C—R². In some embodiments, W² is chosen from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂, In some embodiments, W² is chosen from C—H, C—F, C-D, C—CF₂H, C-CD₃, and C—CF₃.

In embodiments where W² is C—R², R² is chosen from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino.

In some embodiments where W² is C—R², R² is chosen from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo or carboxamide. In some embodiments, R² is chosen from hydrogen, trifluoromethyl, methyl, ethyl, methoxy, trifluoromethoxy, amino, hydroxy, nitrile, halo or carboxamide. In some embodiments, R² is chosen from hydrogen, halo, and perfluoro(C₁-C₃)alkyl.

In some embodiments of formulae (I), (II), (III), and (IVA)-(IVE), W³ is N. In other embodiments of formulae (I), (II), (III), and (IVA)-(IVE), W³ is C—R³. In some embodiments, W³ is chosen from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br. In some embodiments, W³ is chosen from N, C—H, C—NH₂, C—F, C—CF₃, C—CF₂H, C-CD₃, and C-D.

In some embodiments where W³ is C—R³, R³ is chosen from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino.

In some embodiments where W³ is C—R³, R³ is chosen from hydrogen, perfluoro(C₁-C₄)alkyl, (C₁-C₄)alkyl, (Ct-C₄)alkoxy, perfluoro(C₁-C₄)alkoxy, amino, hydroxy, nitrile, halo or carboxamide. In some embodiments where W³ is C—R³, R³ is chosen from hydrogen, trifluoromethyl, methyl, ethyl, methoxy, trifluoromethoxy, amino, hydroxy, nitrile, halo or carboxamide. In some embodiments where W³ is C—R³, R³ is chosen from H, halo, and perfluoro(C₁-C₃)alkyl.

In some embodiments of formulae (I), (II), (III), and (IVA)-(IVE), W⁴ is N. In other embodiments of formulae (I), (II), (III), and (IVA)-(IVE), W⁴ is C—R⁴. In some embodiments, W⁴ is chosen from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br. In some embodiments, W⁴ is chosen from N, C—H, C—NH₂, C—F, C—CF₃, C—CF₂H, C-CD₃, and C-D.

In some embodiments where W⁴ is C—R⁴, R⁴ is chosen from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (Ct-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino.

In some embodiments where W⁴ is C—R⁴, R⁴ is chosen from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo or carboxamide. In some embodiments where W⁴ is C—R⁴, R⁴ is chosen from hydrogen, trifluoromethyl, methyl, ethyl, methoxy, trifluoromethoxy, amino, hydroxy, nitrile, halo or carboxamide. In some embodiments where W⁴ is C—R⁴, R⁴ is chosen from H, halo, and perfluoro(C₁-C₃)alkyl.

In some embodiments of formulae (I), (II), (III), and (IVA)-(IVE), W⁵ is N. In other embodiments of formulae (I), (II), (III), and (IVA)-(IVE), W⁵ is C—R⁵. In some embodiments, W⁵ is chosen from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂. In some embodiments, W⁵ is chosen from C—H, C—F, C-D, C—CF₂H, C-CD₃, and C—CF₃.

In embodiments where W⁵ is C—R⁵, R⁵ is chosen from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino.

In some embodiments where W⁵ is C—R⁵, R⁵ is chosen from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo or carboxamide. In some embodiments, R⁵ is chosen from hydrogen, trifluoromethyl, methyl, ethyl, methoxy, trifluoromethoxy, amino, hydroxy, nitrile, halo or carboxamide. In some embodiments, R⁵ is chosen from hydrogen, halo, and perfluoro(C₁-C₃)alkyl.

In some embodiments of formulae (II) and (III), W⁶ is N. In other embodiments of formulae (II) and (III), W⁶ is C—R⁶, such as C—H.

In some embodiments where W⁶ is C—R⁶, R⁶ is chosen from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino.

In some embodiments of formulae (II) and (III), W⁷ is N. In other embodiments of formulae (II) and (III), W⁷ is C—R⁷. In some embodiments where W⁷ is C—R⁷, R⁷ is chosen from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy. In some embodiments where W⁷ is C—R⁷, R⁷ is hydrogen or (C₃-C₄)alkyl.

In some embodiments of formulae (11) and (I11), W⁸ is N. In other embodiments of formulae (II) and (III), W¹ is C—R⁸. In some embodiments where W⁸ is C—R⁸, R⁸ is chosen from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino. In some embodiments where W⁸ is C—R⁸, R⁸ is chosen from H, (C₁-C₄)alkyl, amino, (C₁-C₄)alkoxy, halo(C₁-C₄)alkoxy, and hydroxy. In some embodiments where W⁸ is C—R⁸, R⁸ is chosen from H, tert-butyl, amino, and methoxy. In some embodiments R^{s} is tert-butyl when W⁷ is N or R⁷ is hydrogen.

In some embodiments of formulae (I), (III), and (IVA)-(IVE) when W¹ is CR⁵⁰, W⁹ is N. In other embodiments of formulae (I), (III), and (IVA)-(IVE) when W¹ is NR¹, O, or S, W⁹ is C.

In some embodiments of formulae (II) and (III), R⁹ is chosen from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy. In some embodiments when R⁷ is hydrogen and R⁸ is H, R⁹ is tert-butyl.

In some embodiments of formulae (II) and (III), R¹⁰ is chosen from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino. In some embodiments, R¹⁰ is hydrogen.

In some embodiments of formula (I), Ar is

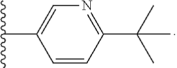 [SMILES: *c1ccc(C(C)(C)C)nc1]

In some embodiments of formula (I), Ar is

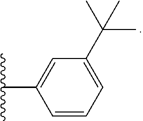 [SMILES: *c1cccc(C(C)(C)C)c1]

In some embodiments of formula (I), Ar is

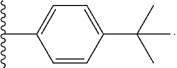 [SMILES: *c1ccc(C(C)(C)C)cc1]

In some embodiments of formula (I), Ar is

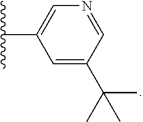 [SMILES: *c1cncc(C(C)(C)C)c1]

In some embodiments of formula (I), Ar is

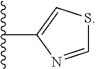 [SMILES: *c1cscn1]

In some embodiments, the compound of formula (I) is selected from a compound of Table 1:

TABLE 1
Compounds of Formula (I)

| No. | Structure/Name | No. | Structure/Name |
|---|---|---|---|
| 1 | 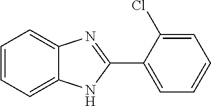 [SMILES: Clc1ccccc1-c1nc2ccccc2[nH]1] | 2 | 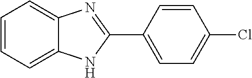 [SMILES: Clc1ccc(-c2nc3ccccc3[nH]2)cc1] |
| | 2-(2-Chlorophenyl)-1H- | | 2-(4-Chlorophenyl)-1H- |
| | benzo[d]imidazole | | benzo[d]imidazole |
| 3 | 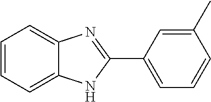 [SMILES: Cc1cccc(-c2nc3ccccc3[nH]2)c1] | 4 | 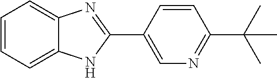 [SMILES: CC(C)(C)c1ccc(-c2nc3ccccc3[nH]2)cn1] |
| | 2-(3-Methylphenyl)-1H- | | 2-(6-tert-Butyl-3-pyridyl)-1H- |
| | benzo[d]imidazole | | benzo[d]imidazole |
| 5 | 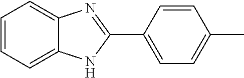 [SMILES: Cc1ccc(-c2nc3ccccc3[nH]2)cc1] | 6 | 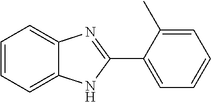 [SMILES: Cc1ccccc1-c1nc2ccccc2[nH]1] |
| | 2-(4-Methylphenyl)-1H- | | 2-(2-Methylpheny1)-1H- |
| | benzo[d]imidazole | | benzo[d]imidazole |
| 7 | 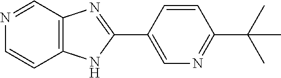 [SMILES: CC(C)(C)c1ccc(-c2nc3cnccc3[nH]2)cn1] | 8 | 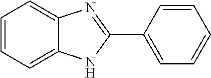 [SMILES: c1ccc(-c2nc3ccccc3[nH]2)cc1] |
| | 2-(6-tert-Butyl-3-pyridyl)-1H- | | 2-(Phenyl)-1H-benzo[d]imidazole |
| | imidazo[4,5-c]-pyridine | | |
| 9 | 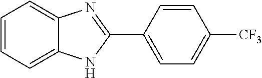 [SMILES: FC(F)(F)c1ccc(-c2nc3ccccc3[nH]2)cc1] | 10 | 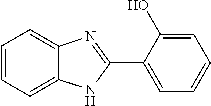 [SMILES: Oc1ccccc1-c1nc2ccccc2[nH]1] |
| | 2-(4-Trifluoro-methylphenyl)-1H- | | 2-(2-Hydroxy-phenyl)-1H- |
| | benzo[d]imidazole | | benzo[d]imidazole |
| 11 | 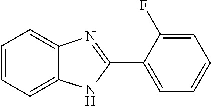 [SMILES: Fc1ccccc1-c1nc2ccccc2[nH]1] | 12 | 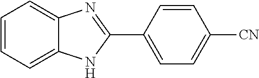 [SMILES: N#Cc1ccc(-c2nc3ccccc3[nH]2)cc1] |
| | 2-(2-Fluorophenyl)-1H- | | 2-(4-Cyanophenyl)-1H- |
| | benzo[d]imidazole | | benzo[d]imidazole |
| 13 | 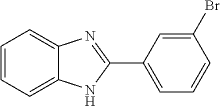 [SMILES: Brc1cccc(-c2nc3ccccc3[nH]2)c1] | 14 | 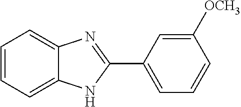 [SMILES: COc1cccc(-c2nc3ccccc3[nH]2)c1] |
| | 2-(3-Bromophenyl)-1H- | | 2-(3-Methoxy-phenyl)-1H- |
| | benzo[d]imidazole | | benzo[d]imidazole |
| 15 | 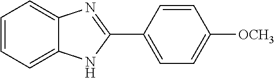 [SMILES: COc1ccc(-c2nc3ccccc3[nH]2)cc1] | 16 | 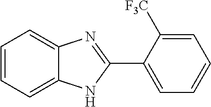 [SMILES: FC(F)(F)c1ccccc1-c1nc2ccccc2[nH]1] |
| | 2-(4-Methoxy-phenyl)-1H- | | 2-(2-Trifluoro-methylphenyl)-1H- |
| | benzo[d]imidazole | | benzo[d]imidazole |
| 17 | 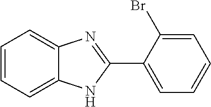 [SMILES: Brc1ccccc1-c1nc2ccccc2[nH]1] | 18 | 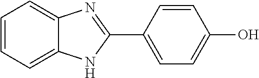 [SMILES: Oc1ccc(-c2nc3ccccc3[nH]2)cc1] |
| | 2-(2-Bromophenyl)-1H- | | 2-(4-Hydroxy-phenyl)-1H- |
| | benzo[d]imidazole | | benzo[d]imidazole |
| 19 | 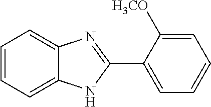 [SMILES: COc1ccccc1-c1nc2ccccc2[nH]1] | 20 | 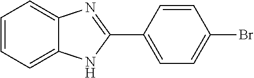 [SMILES: Brc1ccc(-c2nc3ccccc3[nH]2)cc1] |
| | 2-(2-Methoxy-phenyl)-1H- | | 2-(4-Bromophenyl)-1H- |
| | benzo[d]imidazole | | benzo[d]imidazole |
| 21 | 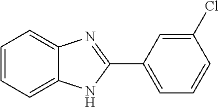 [SMILES: Clc1cccc(-c2nc3ccccc3[nH]2)c1] | 22 | 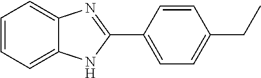 [SMILES: CCc1ccc(-c2nc3ccccc3[nH]2)cc1] |
| | 2-(3-Chlorophenyl)-1H- | | 2-(4-Ethylphenyl)-1H- |
| | benzo[d]imidazole | | benzo[d]imidazole |
| 23 | 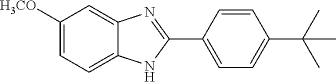 [SMILES: COc1ccc2[nH]c(-c3ccc(C(C)(C)C)cc3)nc2c1] | 24 | 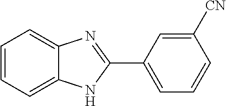 [SMILES: N#Cc1cccc(-c2nc3ccccc3[nH]2)c1] |
| | 2-(4-tert-Butylphenyl)-5-methoxy- | | 2-(3-Cyanophenyl)-1H- |
| | 1H-benzo[d]imidazole | | benzo[d]imidazole |
| 25 | 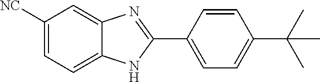 [SMILES: CC(C)(C)c1ccc(-c2nc3cc(C#N)ccc3[nH]2)cc1] | 26 | 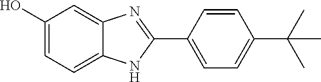 [SMILES: CC(C)(C)c1ccc(-c2nc3cc(O)ccc3[nH]2)cc1] |
| | 2-(4-tert-Butylphenyl)-1H- | | 2-(4-tert-Butylphenyl)-1H- |
| | benzo[d]imidazole-5-carbonitrile | | benzo[d]imidazol-5-ol |
| 27 | 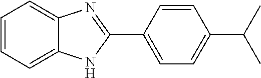 [SMILES: CC(C)c1ccc(-c2nc3ccccc3[nH]2)cc1] | 28 | 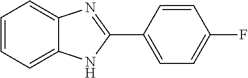 [SMILES: Fc1ccc(-c2nc3ccccc3[nH]2)cc1] |
| | 2-(4-Isopropylphenyl)-1H- | | 2-(4-Fluorophenyl)-1H- |
| | benzo[d]imidazole | | benzo[d]imidazole |
| 29 | 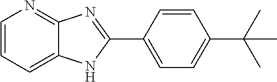 [SMILES: CC(C)(C)c1ccc(-c2nc3ncccc3[nH]2)cc1] | 30 | 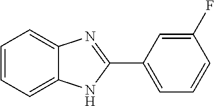 [SMILES: Fc1cccc(-c2nc3ccccc3[nH]2)c1] |
| | 2-(4-tert-Butylphenyl)-1H- | | 2-(3-Fluorophenyl)-1H- |
| | imidazo[4,5-b]pyridine | | benzo[d]imidazole |
| 31 | 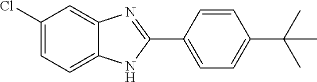 [SMILES: CC(C)(C)c1ccc(-c2nc3cc(Cl)ccc3[nH]2)cc1] | 32 | 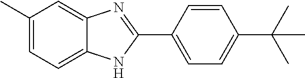 [SMILES: Cc1ccc2[nH]c(-c3ccc(C(C)(C)C)cc3)nc2c1] |
| | 2-(4-tert-Butylphenyl)-5-chloro- | | 2-(4-tert-Butylphenyl)-5-methyl- |
| | 1H-benzo[d]imidazole | | 1H-benzo[d]imidazole |
| 33 | 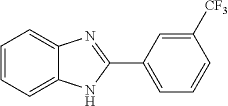 [SMILES: FC(F)(F)c1cccc(-c2nc3ccccc3[nH]2)c1] | 34 | 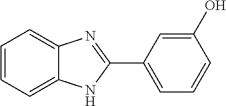 [SMILES: Oc1cccc(-c2nc3ccccc3[nH]2)c1] |
| | 2-(3-Trifluoromethylphenyl)-1H- | | 2-(3-Hydroxyphenyl)-1H- |
| | benzo[d]imidazole | | benzo[d]imidazole |
| 35 | 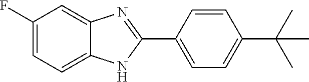 [SMILES: CC(C)(C)c1ccc(-c2nc3cc(F)ccc3[nH]2)cc1] | 36 | 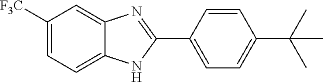 [SMILES: CC(C)(C)c1ccc(-c2nc3cc(C(F)(F)F)ccc3[nH]2)cc1] |
| | 2-(4-tert-Butylphenyl)-5-fluoro- | | (4-tert-Butylphenyl)-5- |
| | 1H-benzo[d]imidazole | | (trifluoromethyl)-1H- |
| | | | benzo[d]imidazole |
| 37 | 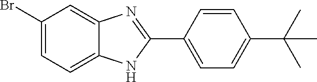 [SMILES: CC(C)(C)c1ccc(-c2nc3cc(Br)ccc3[nH]2)cc1] | 38 | 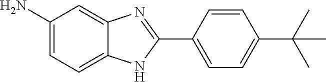 [SMILES: CC(C)(C)c1ccc(-c2nc3cc(N)ccc3[nH]2)cc1] |
| | 2-(4-tert-Butylphenyl)-5-bromo- | | 2-(4-tert-Butylphenyl)-1H- |
| | 1H-benzo[d]imidazole | | benzo[d]imidazol-5-amine |
| 39 | 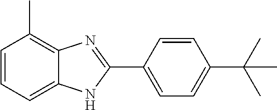 [SMILES: Cc1cccc2[nH]c(-c3ccc(C(C)(C)C)cc3)nc12] | 40 | 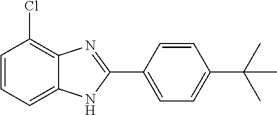 [SMILES: CC(C)(C)c1ccc(-c2nc3c(Cl)cccc3[nH]2)cc1] |
| | 2-(4-tert-Butylphenyl)-4-methyl- | | 2-(4-tert-Butylphenyl)-4-chloro- |
| | 1H-benzo[d]imidazole | | 1H-benzo[d]imidazole |
| 41 | 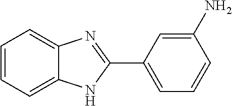 [SMILES: Nc1cccc(-c2nc3ccccc3[nH]2)c1] | 42 | 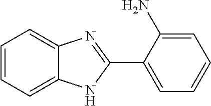 [SMILES: Nc1ccccc1-c1nc2ccccc2[nH]1] |
| | 2-(3-Aminophenyl)-1H- | | 2-(2-Aminophenyl)-1H- |
| | benzo[d]imidazole | | benzo[d]imidazole |
| 43 | 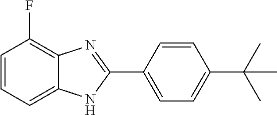 [SMILES: CC(C)(C)c1ccc(-c2nc3c(F)cccc3[nH]2)cc1] | 44 | 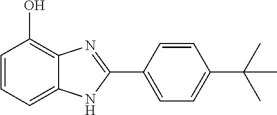 [SMILES: CC(C)(C)c1ccc(-c2nc3c(O)cccc3[nH]2)cc1] |
| | 2-(4-tert-Butylphenyl)-4-fluoro- | | 2-(4-tert-Butylphenyl)-1H- |
| | 1H-benzo[d]imidazole | | benzo[d]imidazol-4-ol |
| 45 | 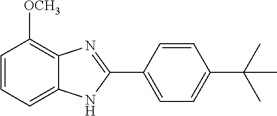 [SMILES: COc1cccc2[nH]c(-c3ccc(C(C)(C)C)cc3)nc12] | 46 | 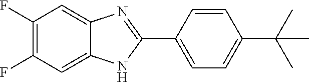 [SMILES: CC(C)(C)c1ccc(-c2nc3cc(F)c(F)cc3[nH]2)cc1] |
| | 2-(4-tert-Butylphenyl)-4-methoxy- | | 2-(4-tert-Butylphenyl)-5,6- |
| | 1H-benzo[d]imidazole | | difluoro-1H-benzo[d]imidazole |
| 47 | 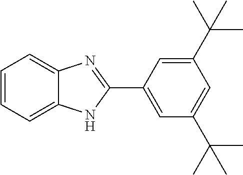 [SMILES: CC(C)(C)c1cc(-c2nc3ccccc3[nH]2)cc(C(C)(C)C)c1] | 48 | 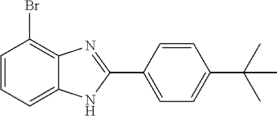 [SMILES: CC(C)(C)c1ccc(-c2nc3c(Br)cccc3[nH]2)cc1] |
| | 2-(3,5-di-tert-Butylphenyl)-1H- | | 2-(4-tert-Butylphenyl)-4-bromo- |
| | benzo[d]imidazole | | 1H-benzo[d]imidazole |
| 49 | 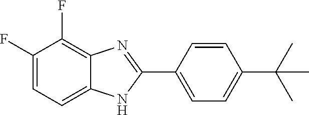 [SMILES: CC(C)(C)c1ccc(-c2nc3c(F)c(F)ccc3[nH]2)cc1] | 50 | 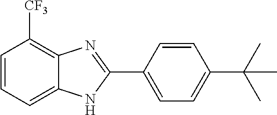 [SMILES: CC(C)(C)c1ccc(-c2nc3c(C(F)(F)F)cccc3[nH]2)cc1] |
| | 2-(4-tert-Butylphenyl)-4,5- | | 2-(4-tert-Butylphenyl)-4- |
| | difluoro-1H-benzo[d]imidazole | | (trifluoromethyl)-1H- |
| | | | benzo[d]imidazole |
| 51 | 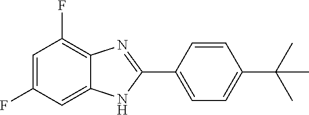 [SMILES: CC(C)(C)c1ccc(-c2nc3c(F)cc(F)cc3[nH]2)cc1] | 52 | 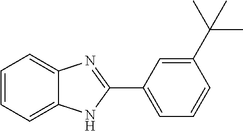 [SMILES: CC(C)(C)c1cccc(-c2nc3ccccc3[nH]2)c1] |
| | 2-(4-tert-Butylphenyl)-4,6- | | 2-(3-tert-Butylphenyl)-1H- |
| | difluoro-1H-benzo[d]imidazole | | benzo[d]imidazole |
| 53 | 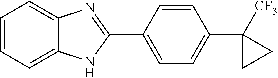 [SMILES: FC(F)(F)C1(c2ccc(-c3nc4ccccc4[nH]3)cc2)CC1] | 54 | 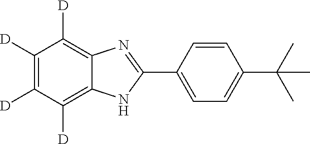 [SMILES: [2H]c1c([2H])c([2H])c2[nH]c(-c3ccc(C(C)(C)C)cc3)nc2c1[2H]] |
| | 2-[4-[1-(Trifluoromethyl)- | | 2-(4-tert-Butylphenyl)-4,5,6,7- |
| | cyclopropyl]phenyl]-1H- | | tetradeuterio-1H- |
| | benzo[d]imidazole | | benzo[d]imidazole |
| 55 | 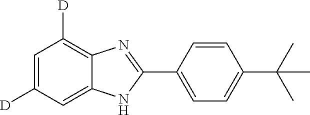 [SMILES: [2H]c1cc([2H])c2nc(-c3ccc(C(C)(C)C)cc3)[nH]c2c1] | 56 | 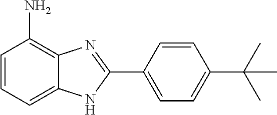 [SMILES: CC(C)(C)c1ccc(-c2nc3c(N)cccc3[nH]2)cc1] |
| | 2-(4-tert-Butylphenyl)-4,6- | | 2-(4-tert-Butylphenyl)-1H- |
| | dideuterio-1H-benzo[d]imidazole | | benzo[d]imidazol-4-amine |
| 57 | 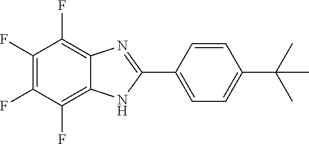 [SMILES: CC(C)(C)c1ccc(-c2nc3c(F)c(F)c(F)c(F)c3[nH]2)cc1] | 58 | 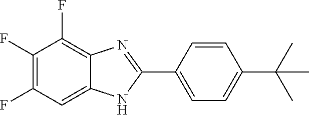 [SMILES: CC(C)(C)c1ccc(-c2nc3c(F)c(F)c(F)cc3[nH]2)cc1] |
| | 2-(4-tert-Butylphenyl)-4,5,6,7- | | 2-(4-tert-Butylphenyl)-4,5,6- |
| | tetrafluoro-1H-benzo[d]imidazole | | trifluoro-1H-benzo[d]imidazole |
| 59 | 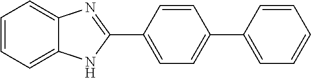 [SMILES: c1ccc(-c2ccc(-c3nc4ccccc4[nH]3)cc2)cc1] | 60 | 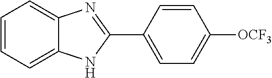 [SMILES: FC(F)(F)Oc1ccc(-c2nc3ccccc3[nH]2)cc1] |
| | 2-(4-Biphenyl)-1H- | | 2-(4-Trifluoromethoxyphenyl)- |
| | benzo[d]imidazole | | 1H-benzo[d]imidazole |
| 61 | 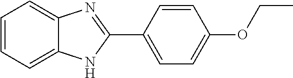 [SMILES: CCOc1ccc(-c2nc3ccccc3[nH]2)cc1] | 62 | 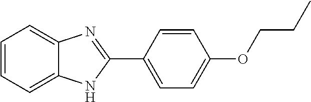 [SMILES: CCCOc1ccc(-c2nc3ccccc3[nH]2)cc1] |
| | 2-(4-Ethoxyphenyl)-1H- | | 2-(4-Propoxyphenyl)-1H- |
| | benzo[d]imidazole | | benzo[d]imidazole |
| 63 | 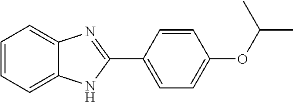 [SMILES: CC(C)Oc1ccc(-c2nc3ccccc3[nH]2)cc1] | 64 | 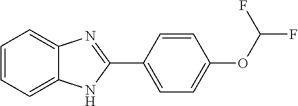 [SMILES: FC(F)Oc1ccc(-c2nc3ccccc3[nH]2)cc1] |
| | 2-(4-Isopropoxyphenyl)-1H- | | 2-(4-(Difluoromethoxy)phenyl)- |
| | benzo[d]imidazole | | 1H-benzo[d]imidazole |
| 65 | 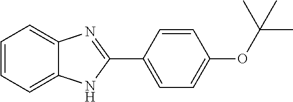 [SMILES: CC(C)(C)Oc1ccc(-c2nc3ccccc3[nH]2)cc1] | 66 | 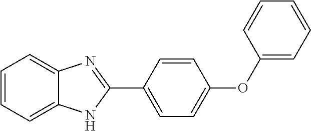 [SMILES: c1ccc(Oc2ccc(-c3nc4ccccc4[nH]3)cc2)cc1] |
| | 2-(4-tert-Butoxyphenyl)-1H- | | 2-(4-Phenoxyphenyl)-1H- |
| | benzo[d]imidazole | | benzo[d]imidazole |
| 67 | 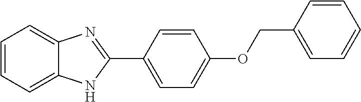 [SMILES: c1ccc(COc2ccc(-c3nc4ccccc4[nH]3)cc2)cc1] | 68 | 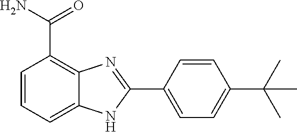 [SMILES: CC(C)(C)c1ccc(-c2nc3c(C(N)=O)cccc3[nH]2)cc1] |
| | 2-(4-Benzyloxyphenyl)-1H- | | 2-(4-tert-Butylphenyl)-1H- |
| | benzo[d]imidazole | | benzo[d]imidazol-4-carboxamide |
| 69 | 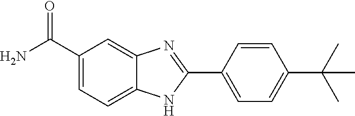 [SMILES: CC(C)(C)c1ccc(-c2nc3cc(C(N)=O)ccc3[nH]2)cc1] | 70 | 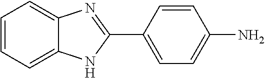 [SMILES: Nc1ccc(-c2nc3ccccc3[nH]2)cc1] |
| | 2-(4-tert-Butylphenyl)-1H- | | 2-(4-Aminophenyl)-1H- |
| | benzo[d]imidazol-5-carboxamide | | benzo[d]imidazole |
| 71 | 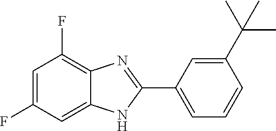 [SMILES: CC(C)(C)c1cccc(-c2nc3c(F)cc(F)cc3[nH]2)c1] | 72 | 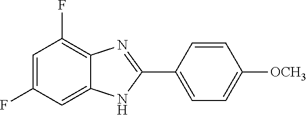 [SMILES: COc1ccc(-c2nc3c(F)cc(F)cc3[nH]2)cc1] |
| | 2-(3-tert-Butylphenyl)-4,6- | | 4,6-Difluoro-2-(4- |
| | difluoro-1H-benzo[d]imidazole | | methoxyphenyl)-1H- |
| | | | benzo[d]imidazole |
| 73 | 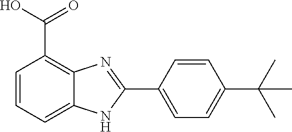 [SMILES: CC(C)(C)c1ccc(-c2nc3c(C(=O)O)cccc3[nH]2)cc1] | 74 | 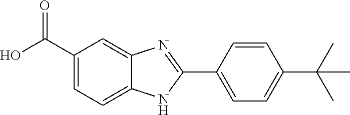 [SMILES: CC(C)(C)c1ccc(-c2nc3cc(C(=O)O)ccc3[nH]2)cc1] |
| | 2-(4-(tert-Butyl)phenyl)-1H- | | 2-(4-(tert-Butyl)phenyl)-1H- |
| | benzo[d]imidazole-4-carboxylic | | benzo[d]imidazole-5-carboxylic |
| | acid | | acid |
| 75 | 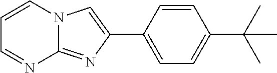 [SMILES: CC(C)(C)c1ccc(-c2cn3cccnc3n2)cc1] | 76 | 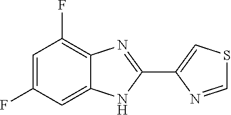 [SMILES: Fc1cc(F)c2nc(-c3cscn3)[nH]c2c1] |
| | Butyl)phenyl)imidazo[1,2- | | 4-(4,6-Difluoro-1H- |
| | alpyrimidinc | | benzo[d]imidazol-2-yl)thiazole |

In some embodiments of formula (I), the compound is selected from 2-(4-(tert-Butyl)phenyl)-1H-imidazo[4,5-c]pyridine; 2-(4-tert-Butylphenyl)-1,3-benzothiazole; 2-(4-tert-Butylphenyl)-1,3-benzoxazole; 2-(4-tert-Butylphenyl)imidazo[1,2-a]pyridine; 2-(2-Chlorophenyl)-1H-benzo[d]imidazole; 2-(4-Chlorophenyl)-1H-benzo[d]imidazole; 2-(3-Methylphenyl)-1H-benzo[d]imidazole; 2-(6-tert-Butyl-3-pyridyl)-1H-benzo[d]imidazole; 2-(4-Methylphenyl)-1H-benzo[d]imidazole; 2-(2-Methylphenyl)-1H-benzo[d]imidazole; 2-(6-tert-Butyl-3-pyridyl)-1H-imidazo[4,5-c]-pyridine; 2-(Phenyl)-1H-benzo[d]imidazole; 2-(4-Trifluoro-methylphenyl)-1H-benzo[d]imidazole; 2-(2-Hydroxy-phenyl)-1H-benzo[d]imidazole; 2-(2-Fluorophenyl)-1H-benzo[d]imidazole; 2-(4-Cyanophenyl)-1H-benzo[d]imidazole; 2-(3-Bromophenyl)-1H-benzo[d]imidazole; 2-(3-Methoxy-phenyl)-1H-benzo[d]imidazole; 2-(4-Methoxy-phenyl)-1H-benzo[d]imidazole; 2-(2-Trifluoro-methylphenyl)-1H-benzo[d]imidazole; 2-(2-Bromophenyl)-1H-benzo[d]imidazole; 2-(4-Hydroxy-phenyl)-1H-benzo[d]imidazole; 2-(2-Methoxy-phenyl)-1H-benzo[d]imidazole; 2-(4-Bromophenyl)-1H-benzo[d]imidazole; 2-(3-Chlorophenyl)-1H-benzo[d]imidazole; 2-(4-Ethylphenyl)-1H-benzo[d]imidazole; 2-(4-tert-Butylphenyl)-5-methoxy-1H-benzo[d]imidazole; 2-(3-Cyanophenyl)-1H-benzo[d]imidazole; 2-(4-tert-Butylphenyl)-1H-benzo[d]imidazole-5-carbonitrile; 2-(4-tert-Butylphenyl)-1H-benzo[d]imidazol-5-ol; 2-(4-Isopropylphenyl)-1H-benzo[d]imidazole; 2-(4-Fluorophenyl)-1H-benzo[d]imidazole; 2-(4-tert-Butylphenyl)-1H-imidazo[4,5-b]pyridine; 2-(3-Fluorophenyl)-1H-benzo[d]imidazole; 2-(4-tert-Butylphenyl)-5-chloro-1H-benzo[d]imidazole; 2-(4-tert-Butylphenyl)-5-methyl-1H-benzo[d]imidazole; 2-(3-Trifluoromethylphenyl)-1H-benzo[d]imidazole; 2-(3-Hydroxyphenyl)-1H-benzo[d]imidazole; 2-(4-tert-Butylphenyl)-5-fluoro-1H-benzo[d]imidazole; 2-(4-tert-Butylphenyl)-5-(trifluoromethyl)-1H-benzo[d]imidazole; 2-(4-tert-Butylphenyl)-5-bromo-1H-benzo[d]imidazole; 2-(4-tert-Butylphenyl)-1H-benzo[d]imidazol-5-amine; 2-(4-tert-Butylphenyl)-4-methyl-1H-benzo[d]imidazole; 2-(4-tert-Butylphenyl)-4-chloro-1H-benzo[d]imidazole; 2-(3-Aminophenyl)-1H-benzo[d]imidazole; 2-(2-Aminophenyl)-1H-benzo[d]imidazole; 2-(4-tert-Butylphenyl)-4-fluoro-1H-benzo[d]imidazole; 2-(4-tert-Butylphenyl)-1H-benzo[d]imidazol-4-ol; 2-(4-tert-Butylphenyl)-4-methoxy-1H-benzo[d]imidazole; 2-(4-tert-Butylphenyl)-5,6-difluoro-1H-benzo[d]imidazole; 2-(3,5-di-tert-Butylphenyl)-1H-benzo[d]imidazole; 2-(4-tert-Butylphenyl)-4-bromo-1H-benzo[d]imidazole; 2-(4-tert-Butylphenyl)-4,5-difluoro-1H-benzo[d]imidazole; 2-(4-tert-Butylphenyl)-4-(trifluoromethyl)-1H-benzo[d]imidazole; 2-(4-tert-Butylphenyl)-4,6-difluoro-1H-benzo[d]imidazole; 2-(3-tert-Butylphenyl)-1H-benzo[d]imidazole; 2-[4-[1-(Trifluoromethyl)-cyclopropyl]phenyl]-1H-benzo[d]imidazole; 2-(4-tert-Butylphenyl)-4,5,6,7-tetradeuterio-1H-benzo[d]imidazole; 2-(4-tert-Butylphenyl)-4,6-dideuterio-1H-benzo[d]imidazole; 2-(4-tert-Butylphenyl)-1H-benzo[d]imidazol-4-amine; 2-(4-tert-Butylphenyl)-4,5,6,7-tetrafluoro-1H-benzo[d]imidazole; 2-(4-tert-Butylphenyl)-4,5,6-trifluoro-1H-benzo[d]imidazole; 2-(4-Biphenyl)-1H-benzo[d]imidazole; 2-(4-Trifluoromethoxyphenyl)-1H-benzo[d]imidazole; 2-(4-Ethoxyphenyl)-1H-benzo[d]imidazole; 2-(4-Propoxyphenyl)-1H-benzo[d]imidazole; 2-(4-Isopropoxyphenyl)-1H-benzo[d]imidazole; 2-(4-(Difluoromethoxy)phenyl)-1H-benzo[d]imidazole; 2-(4-tert-Butoxyphenyl)-1H-benzo[d]imidazole; 2-(4-Phenoxyphenyl)-1H-benzo[d]imidazole; 2-(4-Benzyloxyphenyl)-1H-benzo[d]imidazole; 2-(4-tert-Butylphenyl)-1H-benzo[d]imidazol-4-carboxamide; 2-(4-tert-Butylphenyl)-1H-benzo[d]imidazol-5-carboxamide; 2-(4-Aminophenyl)-1H-benzo[d]imidazole; 2-(3-tert-Butylphenyl)-4,6-difluoro-1H-benzo[d]imidazole; 4,6-Difluoro-2-(4-methoxyphenyl)-1H-benzo[d]imidazole; 2-(4-(tert-Butyl)phenyl)-1H-benzo[d]imidazole-4-carboxylic acid; 2-(4-(tert-Butyl)phenyl)-1H-benzo[d]imidazole-5-carboxylic acid; 2-(4-(tert-Butyl)phenyl)imidazo[1,2-a]pyrimidine; and 4-(4,6-Difluoro-1H-benzo[d]imidazol-2-yl)thiazole.

In some embodiments, the compound of formula (1) is 2-(4-tert-butylphenyl)-1H-benzo[d]imidazol-5-ol (Compound 26) having the formula:

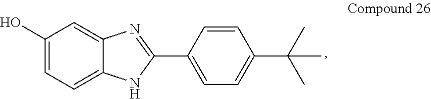 [SMILES: CC(C)(C)c1ccc(-c2nc3cc(O)ccc3[nH]2)cc1]

or a pharmaceutically acceptable salt, solvate, deuterated derivative, or prodrug, thereof.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present disclosure include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salt, solvate, deuterated derivative, or prodrugs include, when appropriate, nontoxic ammonium cations and carboxylate, sulfonate and phosphonate anions attached to alkyl having from 1 to 20 carbon atoms.

While it may be possible for the compounds of formulae (I), (II), (III) and (IVA)-(IVE) to be administered as the raw chemical, in preferred embodiments they are presented as a pharmaceutical composition. In some embodiments, the methods of the present disclosure include administration of a pharmaceutical composition comprising a compound of formula (I), (II), (III) and (IVA)-(IVE), or a pharmaceutically acceptable salt, solvate, deuterated derivative, or prodrug thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof.

The formulations of compounds of formulae (I), (II), (III) and (IVA)-(IVE) include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. In preferred embodiments, the compound is formulated for oral administration. The formulations may conveniently be presented in unit dosage form (e.g., as described herein) and may be prepared by any of the methods well known in the art of pharmacy. All methods include the step of bringing into association a compound of formula (I), (II), (III) and (IVA)-(IVE), or a pharmaceutically acceptable salt, solvate, deuterated derivative, or prodrug thereof (“active ingredient”), with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, tablets or sachets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

### 3.3. Amorphous solid dispersions of 2-(4-tert-butylphenyl)-1H-benzimidazole (TQS-168)

In certain aspects, the methods of treating a hemoglobinopathy and/or for increasing production of HbF in a subject comprise administering a therapeutically effective amount of 2-(4-tert-butylphenyl)-1H-benzimidazole (TQS-168; also known as ZLN-005),

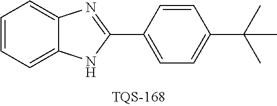 [SMILES: CC(C)(C)c1ccc(-c2nc3ccccc3[nH]2)cc1]

or a pharmaceutically acceptable salt, solvate, deuterated derivative, or prodrug thereof, in an amorphous solid dispersion.

A solid dispersion refers to a system in a solid-state comprising at least two components, wherein one component (e.g., a drug substance) is dispersed throughout the other component or components. In an embodiment, solid dispersions of TQS-168 of the present application comprises TQS-168 or a pharmaceutically acceptable salt, solvate, deuterated derivative, or prodrug, and a pharmaceutically acceptable carrier matrix.

The solid dispersions of TQS-168 may be formed by any conventional technique, e.g., spray drying, co-grinding, hot melt extrusion, freeze drying, rotary evaporation, solvent evaporation, co-precipitation, lyophilization, or any suitable solvent removal process. In some embodiments, the amorphous solid dispersion is prepared by spray drying (e.g., to obtain a spray-dried dispersion (SDD) formulation of TQS-168). In some embodiments, the amorphous solid dispersion is prepared by hot-melt extrusion (e.g., to obtain a hot-melt extrusion (HME) formulation of TQS-168).

The TQS-168 starting material used in the process for preparation of the solid dispersion may be crystalline or amorphous form. Alternatively, it may be obtained in situ from a previous processing step.

In some embodiments, TQS-168 in the solid dispersion obtained is present in an amorphous form.

A solid that is in the “amorphous” solid state form means that it is in a non-crystalline state. Amorphous solids generally possess crystal-like short-range molecular arrangement, but no long-range order of molecular packing as are found in crystalline solids. The solid-state form of a solid, such as the drug substance in the amorphous dispersion, may be determined by Polarized Light Microscopy, X-Ray Powder Diffraction (XPRD), Differential Scanning calorimetry (DSC), or other standard techniques known to those of skill in the art. In some embodiments, the amorphous solid contains TQS-168 in a substantially amorphous solid-state form, e.g., at least about 80% of TQS-168 in the dispersion is in an amorphous form, such as at least about 90% of TQS-168 in the dispersion is in an amorphous form, or at least about 95% of TQS-168 in the dispersion is in amorphous form.

In some embodiments, at least about 90% (e.g., at least 95%, 96%, 97%, 98%, 99%, 99.5%, or even 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of TQS-168 is in amorphous form.

The solid dispersion can be in a single phase such as substitutional or interstitial amorphous solutions; or it can be a two-phase system such as eutectics, amorphous drug and amorphous carrier dispersions. Solid solutions are a resultant single phase upon dispersion of two compounds in each other, at their molecular level.

In some embodiments, the amorphous solid dispersion of TQS-168 includes at least one pharmaceutically acceptable carrier in the carrier matrix.

According to some embodiments of the disclosure, the carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid® 244 FP) and mixtures thereof.

According to the embodiments of the disclosure, the hydroxypropyl methylcellulose acetate succinate (HPMC-AS) comprises various types, such as LF, LG, MF, MG, HF and HG, etc., the first letters L, M and H of the type's names mean the pH level at the beginning of dissolution of HPMC-AS. For example, L refers to low level (e.g., HPMC-AS begins to be dissolved when the pH value is more than 5.5), M refers to middle level (e.g., HPMC-AS begins to be dissolved when the pH value is more than 6.0), H refers to high level (e.g., HPMC-AS begins to be dissolved when the pH value is more than 6.5). The second letters F and G refer to the particle size of HPMC-AS, where F refers to fine powder, and G refers to granular. In some embodiments, the type of HPMC-AS is LF; in some embodiments, the type of HPMC-AS is MF; in some embodiments, the type of HPMC-AS is HG.

More generally, any convenient carrier polymer can find use in subject amorphous solid dispersion formulations. In some embodiments, the carrier polymers include but are not limited to, cellulose acetate phthalate, cellulose acetate trimellitate, cellulose acetate succinate, methyl cellulose phthalate, ethylhydroxymethylcellulose phthalate, hydroxypropylmethylcellulose phthalate (HPMCP), hydroxypropylmethyl cellulose acetate succinate (HPMC-AS), hydroxypropylmethyl cellulose acetate maleate, hydroxypropylmethylcellulose trimellitate, carboxymethylethyl cellulose, polyvinyl butyrate phthalate, polyvinyl acetate phthalate, a methacrylic acid/ethyl acrylate copolymer and a methacrylic acid/methyl methacrylate copolymer. In some embodiments, the polymer is selected from HPMCP, HPMC-AS, hydroxypropylmethyl cellulose acetate maleate and hydroxypropylmethylcellulose trimellitate.

In some embodiments, the carrier polymer is selected from the group consisting of hydroxypropyl methylcellulose, hydroxypropyl cellulose, hydroxyethyl cellulose, polyvinylpyrrolidone (povidone), poly(vinylpyrrolidone/vinylacetate) (copovidone), polyvinylcaprolactam/polyvinylacetate/polyethylene glycol graft copolymer, polyethylene glycol/polyvinyl alcohol graft copolymer, polyethylene oxide, polypropylene oxide, copolymers of ethylene oxide and propylene oxide, polyvinyl alcohol, partially saponified polyvinylalcohol, macrogolglycerol hydroxystearate, polyethylene glycol, and maltodextrins. In some embodiments, the carrier polymer is a copovidone polymer.

In some embodiments, the carrier matrix comprises a copovidone polymer, a povidone polymer, a polyvinyl caprolactam-polyvinyl acetate-polyethylene glycol graft co-polymer, amorphous silicon dioxide (e.g., Syloid® 244 FP), or a mixture thereof.

In some embodiments, carrier matrix comprises a mixture of a polyvinyl caprolactam-polyvinyl acetate-polyethylene glycol graft co-polymer (e.g., soluplus), and amorphous silicon dioxide (e.g., Syloid® 244 FP).

In some embodiments, carrier matrix comprises a mixture of copovidone polymer, a povidone polymer, and a polyvinyl caprolactam-polyvinyl acetate-polyethylene glycol graft co-polymer.

In some embodiments, the amorphous solid dispersion of the TQS-168, and pharmaceutically acceptable carrier matrix have a weight ratio of the TQS-168 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments, TQS-168 and carrier matrix are present in a ratio of from 1:1 to 1:6 (w/w), 1:1 to 1:4 (w/w), such as in the ratio of 1:2 to 1.3 (w/w).

Solid dispersions of the present disclosure optionally may include one or more solubilizers, i.e., additives which increase solubility of the pharmaceutical active ingredient in the solid dispersion or additives which act as pore-forming agents in the solid dispersion. Suitable solubilizers for use in compositions of the present disclosure include mannitol, transcutol, polyvinylalcohol, hydroxypropyl methylcellulose, hydroxypropyl cellulose, methyl cellulose, polyvinylpyrrolidone, glycofurol and transcutol. The concentration of solubilizer ranges from about 0.5% to about 30% w/w of carrier concentration.

The amorphous solid dispersions of the present disclosure optionally may include one or more surfactants. Surfactants are compounds which are capable of improving the wetting of the pharmaceutical active ingredient and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present disclosure include, but not limited to, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, sodium lauryl sulfate, sodium dioctyl sulfosuccinate (DOSS), lecithin, stearylic alcohol, cetostearylic alcohol, cholesterol, polyoxyethylene ricin oil, polyoxyethylene fatty acid glycerides, cremophor RH 40, and the like or combinations thereof. The concentration of surfactant ranges from about 0.1% to about 10% w/w of carrier concentration.

In some embodiments herein, the percentage loading of TQS-168 in the solid dispersion is from 1% to 40% (w/w) (e.g., from 1% to 35%, from 10% to 35%, from 10% to 30%, from 20% to 30%, from 21% to 30%, from 22% to 30%, from 23% to 30%, from 24% to 30%, from 25% to 30%, from 26% to 30%, from 27% to 30%, from 28% to 30%). In some embodiments, the percentage loading of TQS-168 is from 15% to 35% (w/w) (e.g., from 15% to 34%, from 15% to 33%, from 15% to 32%, from 15% to 31%, from 15% to 30%, from 20% to 30%, from 20% to 25%, from 25% to 30%).

In some embodiments, the percentage loading of TQS-168 in the solid dispersion is from 20-30% w/w. In some embodiments, the percentage loading of TQS-168 in the solid dispersion is from 25-30% w/w, such as 25%, 26%, 27%, 28%, 29%, or 30% w/w TQS-168. In some embodiments, the percentage loading of TQS-168 in the solid dispersion is 25% w/w. In some embodiments, the percentage loading of TQS-168 in the solid dispersion is 30% w/w.

In some embodiments herein, the percentage loading of TQS-168 in the solid dispersion is from 1% to 40% (w/w) (e.g., from 1% to 35%, from 10% to 35%, from 10% to 30%, from 20% to 30%, from 21% to 30%, from 22% to 30%, from 23% to 30%, from 24% to 30%, from 25% to 30%, from 26% to 30%, from 27% to 30%, from 28% to 30%, from 30% to 40%, from 35% to 40%). In some embodiments, the percentage loading of TQS-168 is from 15% to 35% (w/w) (e.g., from 15% to 34%, from 15% to 33%, from 15% to 32%, from 15% to 31%, from 15% to 30%, from 20% to 30%, from 20% to 25%, from 25% to 30%).

In some embodiments, the percentage of the pharmaceutically acceptable carrier matrix in the solid dispersion is from 1% to 90% (w/w) (e.g., from 1% to 19%, from 10% to 19%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some embodiments, the percentage of the pharmaceutically acceptable carrier matrix in the solid dispersion is from 60% to 85% (w/w) (e.g., from 60% to 80%, from 60% to 75%, from 60% to 70%, from 65% to 85%, from 65% to 80%, from 65% to 80%, from 65% to 77%, from 65% to 76%, from 65% to 75%, from 66% to 75%, from 66% to 75%, from 67% to 75%, from 68% to 75%, and from 70% to 75%).

In some embodiments, the percentage of the pharmaceutically acceptable carrier matrix in the solid dispersion is from 60% to 85% (w/w) (e.g., from 65% to 80%, from 65% to 75%, and from 70 to 75). In some embodiments, the percentage of the pharmaceutically acceptable carrier matrix in the solid dispersion is from 65% to 80% (w/w). In some embodiments, the percentage of the pharmaceutically acceptable carrier matrix in the solid dispersion is 70% (w/w). In some embodiments, the percentage of the pharmaceutically acceptable carrier matrix in the solid dispersion is 75% (w/w).

In some embodiments, amorphous solid dispersions of TQS-168 are obtained by a spray drying process. Spray dried dispersions are obtained by dissolving drug and the carrier polymer in an organic solvent and then spray-drying the mixture to obtain a spray-dried dispersion (SDD). The formulation and process conditions are chosen so that the solvent quickly evaporates from the droplets, allowing insufficient time for phase separation or crystallization. Accordingly, in some embodiments, there is provided an amorphous solid dispersion comprising TQS-168 and a pharmaceutically acceptable carrier matrix, wherein the amorphous solid dispersion is prepared by spray-drying (e.g., to obtain a spray-dried dispersion (SDD) formulation). In an embodiment there is provided a spray-dried dispersion comprising 10-30% w/w of TQS-168 and 70-90% of a pharmaceutically acceptable carrier matrix, wherein the amorphous solid dispersion is prepared by spray-drying (e.g., to obtain a spray-dried dispersion (SDD) formulation).

The term “spray dried dispersion” (SDD) is a single phase amorphous molecular dispersion of a drug in a polymer matrix. It is a solid solution prepared by dissolving the drug and a polymer in a solvent (e.g., methanol, 2-propanol or the like) and spray drying the solution. The solvent rapidly evaporates from droplets which rapidly solidifies the polymer and drug mixture trapping the drug in amorphous form as an amorphous molecular dispersion.

Accordingly, in an embodiment there is provided a spray-dried dispersion (SDD) formulation comprising TQS-168 and a pharmaceutically acceptable carrier matrix. In an embodiment there is provided a spray-dried dispersion comprising 10-30% w/w of TQS-168 and 70-90% of a pharmaceutically acceptable carrier matrix.

In some embodiments, the percentage loading of TQS-168 in the spray-dried dispersion (SDD) formulation is from 15-40% w/w. In some embodiments, the percentage loading of TQS-168 in the spray-dried dispersion (SDD) formulation is from 20-40% w/w, such as 20%, 21%, 22%, 23%, 24%, 25%, 26%, 27%, 28%, 29%, 30%, 31%, 32%, 33%, 34%, 35%, 36%, 37%, 38%, 39%, or 40% w/w TQS-168. In some embodiments, the percentage loading of TQS-168 in the spray-dried dispersion (SDD) formulation is 30% w/w.

In some embodiments, the percentage of the pharmaceutically acceptable carrier matrix in the spray-dried dispersion (SDD) formulation is from 60% to 85% (w/w) (e.g., from 70% to 80%, and from 70% to 75%). In some embodiments, the percentage of the pharmaceutically acceptable carrier matrix in the spray-dried dispersion (SDD) formulation is from 70% to 75% (w/w), such as 70%, 71%, 72%, 73%, 74%, or 75% (w/w). In some embodiments, the percentage of the pharmaceutically acceptable carrier matrix in the solid dispersion is 70% (w/w).

In some embodiments of the spray-dried dispersion (SDD) formulation, the pharmaceutically acceptable carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid® 244 FP) and mixtures thereof.

In some embodiments of the spray-dried dispersion (SDD) formulation, the pharmaceutically acceptable carrier matrix comprises a polyvinyl caprolactam-polyvinyl acetate-polyethylene glycol graft co-polymer (e.g., soluplus), and amorphous silicon dioxide (e.g., Syloid® 244 FP).

In some embodiments of the spray-dried dispersion (SDD) formulation, the ratio of amorphous silicon dioxide (e.g., Syloid® 244 FP) to polyvinyl caprolactam-polyvinyl acetate-polyethylene glycol graft co-polymer (e.g., soluplus) is from 1:2 to 1:1 (e.g., from 1:2 to 1:1.1, from 1:2 to 1:1.2, from 1.2 to 1:1.3, from 1:2 to 1:1.4, from 1:2 to 1:1.5, from 1:2 to 1:1.6, from 1:2 to 1:1.7, from 1:2 to 1:1.8, from 1:2 to 1:1.9, from 1:1.9 to 1:1, from 1:1.8 to 1:1, from 1:1.7 to 1:1, from 1:1.6 to 1:1, from 1:1.5 to 1:1, from 1:1.6 to 1:1, from 1:1.5 to 1:1, from 1:1.4 to 1:1, from 1:1.3 to 1:1, from 1:1.2 to 1:1, and from 1:1.1 to 1:1). In some embodiments, the ratio of amorphous silicon dioxide (e.g., Syloid® 244 FP) to polyvinyl caprolactam-polyvinyl acetate-polyethylene glycol graft co-polymer (e.g., soluplus) is 1:1.3.

In some embodiments of the spray-dried dispersion (SDD) formulation, the amorphous solid dispersion of the TQS-168, and phannaceutically acceptable carrier matrix have a weight ratio of the TQS-168 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments, TQS-168 and carrier matrix are present in a ratio of from 1:1 to 1:6 (w/w), 1:1 to 1:4 (w/w), such as in the ratio of 1:2 to 1.3 (w/w).

In an embodiment, amorphous solid dispersions of TQS-168 are obtained by hot melt extrusion. The term hot-melt extrusion or hot-melt extruded is used herein to describe a process whereby a composition is heated and/or compressed to a molten (or softened) state and subsequently forced through an orifice in a die where the extruded product is formed into its final shape in which it solidifies upon cooling. The blend is conveyed through one or more heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw fights by which the various components of the powder blend are well mixed and disaggregated. The die can be a dual manifold, multi-manifold or feed-block style die.

In some embodiments, there is provided an amorphous solid dispersion comprising TQS-168 and a pharmaceutically acceptable carrier matrix, wherein the amorphous solid dispersion is prepared by hot-melt extrusion (e.g., to obtain a hot-melt extrusion (HME) formulation). In an embodiment there is provided a spray-dried dispersion comprising 10-30% w/w of TQS-168 and 70-90% of a pharmaceutically acceptable carrier matrix, wherein the amorphous solid dispersion is prepared by hot-melt extrusion (e.g., to obtain a hot-melt extrusion (HME) formulation).

Accordingly, in an embodiment there is provided a hot-melt extrusion (HME) formulation comprising TQS-168 and a pharmaceutically acceptable carrier matrix. In an embodiment there is provided a hot-melt extrusion formulation comprising 10-30% w/w of TQS-168 and 70-90% of a pharmaceutically acceptable carrier matrix.

In some embodiments, the percentage loading of TQS-168 in the hot-melt extrusion (HME) formulation is from 15-30% w/w. In some embodiments, the percentage loading of TQS-168 in the hot-melt extrusion (HME) formulation is from 20-40% w/w, such as 20%, 21%, 22%, 23%, 24%, 25%, 26%, 27%, 28%, 29%, 30%, 31%, 32%, 33%, 34%, 35%, 36%, 37%, 38%, 39%, or 40% w/w TQS-168. In some embodiments, the percentage loading of TQS-168 in hot-melt extrusion (HME) formulation is 25% w/w.

In some embodiments, the percentage of the pharmaceutically acceptable carrier matrix in the hot-melt extrusion (HME) formulation is from 60% to 85% (w/w) (e.g., from 70% to 80%, and from 70% to 75%). In some embodiments, the percentage of the pharmaceutically acceptable carrier matrix in the hot-melt extrusion (HME) formulation is from 70% to 75% (w/w), such as 70%, 71%, 72%, 73%, 74%, or 75%. In some embodiments, the percentage of the pharmaceutically acceptable carrier matrix in the hot-melt extrusion (HME) formulation is 75% (w/w).

In some embodiments of the hot-melt extrusion (HME) formulation, the pharmaceutically acceptable carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid® 244 FP) and mixtures thereof.

In some embodiments of the hot-melt extrusion (HME) formulation, the pharmaceutically acceptable carrier matrix comprises a mixture of copovidone polymer, a povidone polymer, and a polyvinyl caprolactam-polyvinyl acetate-polyethylene glycol graft co-polymer.

In some embodiments of the hot-melt extrusion (HME) formulation, the ratio of copovidone polymer, a povidone polymer, and a polyvinyl caprolactam-polyvinyl acetate-polyethylene glycol graft co-polymer is 1:1:1.

In some embodiments of the hot-melt extrusion (HME) formulation, the amorphous solid dispersion of the TQS-168, and pharmaceutically acceptable carrier matrix have a weight ratio of the TQS-168 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments of the hot-melt extrusion (HME) formulation, TQS-168 and carrier matrix are present in a ratio of from 1:1 to 1:6 (w/w), 1:1 to 1:4 (w/w), such as in the ratio of 1:2 to 1.3 (w/w). 3.4. Pharmaceutical Compositions

In some embodiments, the subject compounds—e.g., compounds of formula (I) and subject amorphous solid dispersions comprising TQS-168—are formulated in pharmaceutical compositions together with one or more pharmaceutically acceptable excipients.

The subject compounds and amorphous solid dispersion may be blended with any one of the excipients described herein (e.g., to form a blended powder or granules) or for filling any one of the dosage forms described herein (e.g., tableting). The compounds and amorphous solid dispersion can optionally be further processed before blending or filling. Exemplary further processing includes spheronizing, pelletizing, milling, injection molding, sieving, and/or calendering.

Compounds and amorphous solid dispersions of the present disclosure can be optionally subjected to a particle size reduction procedure before or after the completion of drying of the product to produce desired particle sizes and distributions. Milling or micronization can be performed to achieve the desired particle sizes or distributions. Equipment that may be used for particle size reduction include, without limitation thereto, ball mills, roller mills, hammer mills, and jet mills.

The compounds and amorphous solid dispersion may be combined with pharmaceutically acceptable excipients to make other pharmaceutical compositions, or a finished dosage form (e.g., as described herein). The one or more additional pharmaceutically acceptable excipients are selected from diluents, binders, disintegrants, lubricants, glidants, surfactants, solubilizers, plasticizers, stabilizing agents, antioxidants, sweeteners, colors, flavors, preservatives, and combinations thereof.

Other pharmaceutically acceptable excipients may include, but are not limited to, diluents, binders, disintegrating agents, surfactants, plasticizers, lubricants, glidants, chelating agents, coating agents and the like or mixtures thereof as extra-granular agents.

In some embodiments, the one or more pharmaceutically acceptable excipients is selected from diluents, binders, disintegrants, lubricants, glidants, surfactants, solubilizers, plasticizers, stabilizing agents, antioxidants, sweeteners, and any combination thereof.

In some embodiments of the pharmaceutical composition, the percentage of compound or amorphous solid dispersion present is from 1% to 90% (w/w) (e.g., from 1% to 19%, from 10% to 19%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some embodiments, the percentage of the compound or amorphous solid dispersion in the subject pharmaceutical composition is from 30% to 50% (w/w) (e.g., from 30% to 48%, from 30% to 45%, from 30% to 42%, from 30% to 40%, from 35% to 50%, from 35% to 48%, from 35% to 45%, from 35% to 44%, from 35% to 43%, from 35% to 42%, from 35% to 41%, from 35% to 40%, from 36% to 40%, and from 37% to 40%).

In some embodiments of the pharmaceutical composition, the percentage of the compound or amorphous solid dispersion present is from 30% to 50% (w/w), such as 35 to 45% (w/w). In some embodiments of the pharmaceutical composition, the percentage of amorphous solid dispersion present is from 35 to 45%, such as 35%, 36%, 37%, 38%, 39%, 40%, 41%, 42%, 43%, 44%, or 45% (w/w). In some embodiments of the pharmaceutical composition, the percentage of amorphous solid dispersion present is 40% (w/w).

### 3.4.1. Excipients

In certain embodiments, the disclosure provides pharmaceutical compositions that include a subject compound of formula (I) or amorphous solid dispersion as described herein, and one or more pharmaceutically acceptable excipients or carriers including but not limited to, inert solid diluents and fillers, diluents, including sterile aqueous solution and various organic solvents, permeation enhancers, solubilizers, surfactants, disintegrants, lubricants, binders, glidants, adjuvants, and combinations thereof suitable for oral administration. Such compositions are prepared in a manner well known in the pharmaceutical art (see, e.g., Remington: The Science and Practice of Pharmacy, 23rd Edition, ISBN-13: 978-0128200070); and Modern Pharmaceutics, Marcel Dekker, Inc., 4th Ed. (G. S. Banker & C. T. Rhodes, Eds.).

The pharmaceutical compositions provided in accordance with the present disclosure can be configured for oral administration. The pharmaceutical compositions may be administered by oral administration. Administration may be via an oral suspension of powder or granules, an oral disintegrating tablet (ODT), or the like. In one embodiment, the subject pharmaceutical composition is in the form of a powder. In one embodiment, the subject pharmaceutical composition is in the form of granules. In one embodiment, the subject pharmaceutical composition is in the form of a powder or granules which has been reconstituted as an oral suspension. In one embodiment, the subject pharmaceutical composition is reconstituted in a food or a beverage. In some embodiments, the pharmaceutical composition is in the form of an oral disintegrating tablet (ODT). In making the pharmaceutical compositions that include the solid described herein, the active ingredient is usually diluted by an excipient and/or enclosed within such a carrier that can be in the form of a sachet, or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases, the immediate release formulation may be coated such that the therapeutic agent is only released once it reached the desired target in the body (e.g., the stomach). In a specific embodiment, the pharmaceutical composition is formulated for immediate release.

The pharmaceutical composition may further comprise pharmaceutical excipients such as fillers or diluents, binders, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises a diluent or a filler, such as a carbohydrate or a protein filler. In some embodiments, the diluent is selected from the group consisting of dicalcium phosphate, cellulose, microcrystalline cellulose, hydroxypropylmethyl cellulose, sodium carboxymethylcellulose, compressible sugars, dibasic calcium phosphate dehydrate, lactose, lactose monohydrate, sucrose, mannitol, sorbital, starch from corn, wheat, rice, potato or other plants, tribasic calcium phosphate, a gum (e.g., Arabic or tragacanth), proteins (e.g., gelatin or collagen) and combinations thereof. In some embodiments of the pharmaceutical composition, the diluent is selected from microcrystalline cellulose, dicalcium phosphate, cellulose, compressible sugars, dibasic calcium phosphate dehydrate, lactose, lactose monohydrate, lactose anhydrous, mannitol, tribasic calcium phosphate, a partially pregelatinized starch and combinations thereof.

In further embodiments, the pharmaceutical composition comprises one or more diluents in an amount from 10 to 70% w/w, or from 20 to 70% w/w, or from 25% to 70% w/w, or from 30 to 70% w/w, or from 35 to 70% w/w, or from 40 to 70% w/w, or from 45 to 65% w/w, or from 45 to 60% w/w, or from 50 to 60% w/w. In some embodiments, one or more diluents is present in an amount of 50 to 60% w/w, such as 50%, 51%, 52%, 53%, 54%, 55%, 56%, 57%, 58%, 59% or 60% w/w. In some embodiments, one or more diluents is present in an amount of 50 to 51% w/w. In some embodiments, one or more diluents is present in an amount of 58 to 59% w/w.

In some embodiments of the pharmaceutical composition, the diluent is microcrystalline cellulose present in an amount of from 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w. In some embodiments of the pharmaceutical composition, the diluent is mannitol present in an amount of 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w. In some embodiments of the pharmaceutical composition, the diluent is a pregelatinized starch (e.g., starch 1,500) present in an amount of 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w.

In some embodiments of the pharmaceutical composition, the diluent used has a combination of properties. For example, in some cases the diluent can also acts as a binder and/or a disintegrant.

In some embodiments, the pharmaceutical composition comprises a disintegrant or solubilizing agent selected from cross-linked polyvinyl pyrrolidone, agar, alginic acid or a salt thereof (e.g., sodium alginate), croscarmellose sodium, crospovidone, modified corn starch, pregelatinized starch, sodium starch glycolate, and combinations thereof.

In certain embodiments, the pharmaceutical composition comprises one or more disintegrants in an amount from 1 to 10% w/w, or from 1 to 9% w/w, or from 1 to 8% w/w, or from 2 to 8% w/w, or from 4 to 6% w/w. In some embodiments, one or more disintegrants is present in an amount of 1%, 2%, 3%, 4%, 5%, 6%, 7%, 8%, 9%, or 10% w/w. In some embodiments, the disintegrant is croscannellose sodium present in an amount of 4 to 6% w/w. In a further specific embodiment, the disintegrant is croscarmellose sodium present in an amount of 5% w/w.

In some embodiments, the pharmaceutical composition comprises a lubricant selected from the group consisting of calcium stearate, magnesium stearate, polyethylene glycol, sodium stearyl fumarate, stearic acid, and combinations thereof.

In some embodiments, the pharmaceutical composition comprises one or more lubricants in an amount from about 0.1 to about 2% w/w, or from about 0.5 to about 1.8% w/w, or from about 0.5 to about 1.5% w/w, or from about 0.5 to about 1.4% w/w, or from about 0.5 to about 1.3% w/w, or from about 0.5 to about 1.2% w/w, or from about 0.5 to about 1.1% w/w. In some embodiments, the lubricant is sodium stearyl fumarate present in an amount of 0.5%, 0.6% w/w, 0.7% w/w, 0.8% w/w, 0.9% w/w, or 1.0% w/w. In some embodiments, the lubricant is sodium stearyl fumarate present in an amount of 1.0% w/w.

In some embodiments, the pharmaceutical composition comprises a glidant selected from the group consisting of colloidal silicon dioxide, talc, and combinations thereof.

In some embodiments, the pharmaceutical composition comprises one or more glidants in an amount from about 0.1 to about 2.5% w/w, or from about 0.5 to about 2.0% w/w, or from about 0.5 to about 1.5% w/w. In specific embodiments, one or more glidants is present in an amount of 0.5%, 0.6%, 0.7%, 0.8%, 0.9%, 1.0%, 1.1%, 1.2%, 1.3%, 1.4%, or 1.5% w/w. In some embodiments, the glidant is colloidal silicon dioxide present in an amount of 1.5% w/w.

In some embodiments, the pharmaceutical composition comprises a sweetener. In some embodiments, the sweetener is an artificial sweetener, such as sucralose. It will be understood than any convenient sweetener can be used in the present disclosure.

In some embodiments, the pharmaceutical composition comprises one or more sweeteners in an amount from 1 to 10% w/w, or from 1 to 9% w/w, or from 1 to 8% w/w, or from 2 to 8% w/w, or from 4 to 6% w/w, or from 1 to 5% w/w, or from 1 to 2.5% w/w. In some embodiments, one or more sweeteners is present in an amount of 1%, 2%, 3%, 4%, 5%, 6%, 7%, 8%, 9%, or 10% w/w. In some embodiments, the sweetener is sucralose present in an amount of from 1.5 to 2.5% w/w. In some embodiments, the sweetener is sucralose present in an amount of from 2 to 3% w/w.

In some embodiments, the pharmaceutical composition comprises one or more surfactants. Suitable surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol-20 dioleate, polyethylene glycol 4-150 mono dilaurate, and polyethylene glycol-20 glyceryl stearate; alcohol-oil transesterification products, for example, polyethylene glycol-6 corn oil; polyglycerized fatty acids, for example, polyglyceryl-6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol-20 sorbitan monooleate and sorbitan monolaurate; polyethylene glycol alkyl ether or phenols, for example, polyethylene glycol-20 cetyl ether and polyethylene glycol-10-100 nonyl phenol; sugar esters, for example, sucrose monopalmitate; polyoxyethylene-polyoxypropylene block copolymers known as “poloxamer;” ionic surfactants, for example, sodium caproate, sodium glycocholate, soy lecithin, sodium stearyl fumarate, propylene glycol alginate, octyl sulfosuccinate disodium, and palmitoyl carnitine; and the like and mixtures thereof. The concentration of surfactant ranges from about 0.5% to about 10% w/w of total composition.

In some embodiments, the pharmaceutical composition may include one or more plasticizers. Suitable plasticizers include polyethylene glycol, propylene glycol, polyethylene oxide, 1,2-butylene glycol, 2,3-butylene glycol, styrene glycol, diethylene glycol, triethylene glycol, tetraethylene glycol and monoisopropyl ether, propylene glycol monoethyl ether, ethylene glycol monoethyl ether, diethylene glycol monoethyl ether, sorbitol lactate, ethyl lactate, butyl lactate, ethyl glycolate, triethyl citrate, acetyl triethyl citrate, tributyl citrate and allyl glycolate. The concentration of plasticizer ranges from about 0.5% to about 10% w/w of total composition.

In some embodiments, the pharmaceutical composition may include a coloring agent. Suitable coloring agents include dyes and pigments such as iron oxide red or yellow, titanium dioxide, talc. The concentration of coloring agent can range from about 0.1% to about 1% w/w of the total composition.

In some embodiments, the pharmaceutical composition may include a chelating agent. Suitable chelating agents include, one or more of, but not limited to ethylenediaminetetraacetic acid (EDTA), disodium EDTA and derivatives thereof, citric acid and derivatives thereof, niacinamide and derivatives thereof, and sodium desoxycholate and the like or mixtures thereof. The concentration of chelating agent can range from about 0.1% to about 1% w/w of total composition.

In some embodiments, the pharmaceutical composition comprises the amorphous solid dispersion (e.g., as described herein), one or more diluents, one or more disintegrants, and one or more additional actives. In some embodiments, the pharmaceutical composition comprises 30-50% w/w of the amorphous solid dispersion, 40-70% w/w of one or more diluents, 1-10% w/w of one or more disintegrants, and up to 100% w/w of one or more additional additives. In some embodiments, the pharmaceutical composition comprises 35-45% w/w of the amorphous solid dispersion, 50-60% w/w of one or more diluents, 4-6% w/w of one or more disintegrants, and up to 100% w/w of one or more additional additives. In some embodiments, one or more additional additives includes a lubricant, a glidant and a sweetener.

In some embodiments, the pharmaceutical composition comprises:

TABLE 2

| Component | % w/w |
|---|---|
| an amorphous solid dispersion of TQS-168 | 30 to 50% |
| (e.g., as described herein) | |
| a diluent | 40 to 70% |
| a disintegrant | 1 to 10% |
| a lubricant | 0.5 to 2% |
| a glidant | 0.5 to 2% |
| a sweetener | 0 to 5% |

In some embodiments, the pharmaceutical composition comprises:

TABLE 3

| Component | % w/w |
|---|---|
| an amorphous solid dispersion of TQS-168 | 35 to 45% |
| (e.g., as described herein) | |
| a diluent | 50 to 60% |
| a disintegrant | 4 to 6% |
| a lubricant | 0.5 to 1.5% |
| a glidant | 1 to 2% |
| a sweetener | 0 to 5% |

In some embodiments, the pharmaceutical composition includes 1 to 5% of a sweetener. In some embodiments, the sweetener is an artificial sweetener. In some embodiments, the sweetener is sucralose.

The pharmaceutical compositions described herein can be formulated with a compound of formula (I), or an amorphous solid dispersion of TQS-168 (e.g., as described herein), as the sole pharmaceutically active ingredient in the composition or can be combined with other active ingredients (e.g., as described herein).

In certain embodiments, the pharmaceutical composition is formulated into one or more suitable pharnaceutical preparations, such as oral suspensions, powders, granules, an oral disintegrating tablet (ODT), sustained release formulations or elixirs in sterile solutions or suspensions for parenteral administration, or as transdermal patch preparation and dry powder inhalers.

Concentrations of the subject compounds in a pharmaceutical composition provided herein will depend on, e.g., the physicochemical characteristics of the compound, the dosage schedule, and amount administered as well as other factors known to those of skill in the art. For example, if the composition comprises a salt of TQS-168 the amount of said salt to be administered and/or to be incorporated into a pharmaceutical composition (i.e., pharmaceutical dosage form) needs to be adjusted to take account of the molecular weight difference between the free base and salt form. For instance, in expressing dose amounts in the label and/or product information of authorized medicinal products comprising a salt form of an active compound that can also be used in free base form, it is customary practice to specify the dose of the free base to which the dose of the salt as used is equivalent.

Pharmaceutical compositions described herein are provided for administration to a subject, for example, humans or animals (e.g., mammals) in unit dosage forms, such as sterile parenteral (e.g., intravenous) suspensions containing suitable quantities of the compounds or pharmaceutically acceptable derivatives thereof. Pharmaceutical compositions are also provided for administration to humans and animals in unit dosage form, including powders, granules, oral or nasal solutions or suspensions and oil-water emulsions containing suitable quantities of the subject compounds or pharmaceutically acceptable derivatives thereof. The subject compounds and amorphous solid dispersions are, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of the subject compound sufficient to produce the desired therapeutic effect, in association with the required pharmaceutical carrier, vehicle or filler. Examples of unit-dose forms include sachets, ampoules and syringes and individually packaged tablets. Unit-dose forms can be administered in fractions or multiples thereof. A multiple-dose form is a plurality of identical unit-dosage forms packaged in a single container to be administered in segregated unit-dose form. Examples of multiple-dose forms include sachets, vials, or bottles. Hence, in specific aspects, multiple dose form is a multiple of unit-doses which are not segregated in packaging.

In some embodiments the subject pharmaceutical composition is formulated as powders, granules, tablets, pills, dragees, capsules, liquids, gels, syrups, slurries, suspensions, and the like, suitablefor ingestion by a subject. In certain cases, the pharmaceutical composition is formulated as a dragee, and dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the dragee coatings for product identification or to characterize the quantity of active compound (i.e., dosage).

In some embodiments, the subject pharmaceutical composition is formulated for oral use as push-fit capsules made of gelatin, as well as soft, sealed capsules made of gelatin having a coating such as glycerol or sorbitol. Push-fit capsules can contain compounds and mixed excipients as described herein, e.g., diluents or binders such as microcrystalline cellulose, lubricants such as talc or magnesium stearate, and optionally stabilizers.

In some embodiments, the subject pharmaceutical composition is formulated for oral use as a powder or granules, the unit doses of which can be individually packaged in sachets. In some embodiments, the powder or granules can contain an amorphous solid dispersion of TQS-168 mixed excipients as described herein, e.g., diluents such as microcrystalline cellulose, disintegrants such as croscarmellose sodium, lubricants such as sodium stearyl fumarate, and, optionally one or more other excipients, such as glidants and sweeteners.

In certain embodiments, the subject compounds and amorphous solid dispersions described herein are in a liquid pharmaceutical formulation. Liquid pharmaceutically administrable formulations can, for example, be prepared by dispersing, or otherwise mixing the active compounds and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (Remington: The Science and Practice of Pharmacy, 23rd Edition, ISBN-13: 978-0128200070) Dosage forms or compositions containing the subject compounds and amorphous solid dispersions in the ranges disclosed herein with the balance made up from non-toxic carrier can be prepared.

In certain embodiments, the pharmaceutical formulations are powders or granules, which can be reconstituted for administration as suspensions, emulsions and other mixtures. They can also be reconstituted and formulated as solids or gels.

In certain embodiments, the pharmaceutical composition is formulated in as a solid dosage form, such as an oral disintegrating tablet, a tablet or a capsule.

In some embodiments, the disclosure relates to pharmaceutical compositions comprising subject compounds or amorphous solid dispersions for oral administration.

### 3.4.2. Pharmaceutical Dosage Forms

In some embodiments, there is provided a pharmaceutical dosage form comprising the pharmaceutical composition described herein. The disclosure provides for powders, granules, oral disintegrating tablets, tablets, pills, capsules, oral suspensions and the like, comprising the pharmaceutical compositions or dosage forms described herein. Accordingly, in some embodiments, the pharmaceutical dosage from is a solid dosage form. In certain cases, the solid dosage form is a powder. In certain cases, the capsule is in the form of granules. In certain embodiments, the solid dosage form is in the form of a power or granules packaged in individually labelled sachets containing a single dose. In certain cases, the dosage form is an oral disintegrating tablet (ODT).

In some embodiments the pharmaceutical dosage the dosage forn is formulated for immediate-release. In some embodiments, the dosage form is formulated for modified-release portion.

As used herein the term “immediate-release” refers to the rapid release of the majority of the therapeutic compound. Particularly useful conditions for immediate-release are release of at least or equal to about 80% of the therapeutic compound within thirty minutes after oral ingestion. The particular immediate-release conditions for a specific therapeutic compound will be recognized or known by one of ordinary skill in the art.

As used herein the term “modified-release” refers to slower release of the majority of the therapeutic compound as compared to immediate release dosage forms. The particular modified-release conditions for a specific therapeutic compound will be recognized or known by one of ordinary skill in the art.

The pharmaceutical compositions are manufactured by processes such as direct compression, wet granulation or dry granulation. In some embodiments, the pharmaceutical compositions are in the form of oral dosage forms, such as solid oral dosage forms, including powders, granules, oral disintegrating tablets (ODT), oral suspensions, and multi-particulates.

In some embodiments, the pharmaceutical composition of the present disclosure is a granulate/particulate material. The granules/particles may be filled into a sachet or compressed into a tablet. The tablet may optionally be coated with an additional enteric polymer or an immediate-release coating.

Moreover, the extrudates/granules of the present disclosure may be formulated into any suitable dosage form, including but not limited to oral suspensions, gels, oral disintegrating tablets (ODTs), tablets, capsules, immediate release formulations, delayed release formulations, controlled release formulations, extended-release formulations, pulsatile release formulations, and mixed immediate and controlled release formulations.

The tablets or pills of the present disclosure may be coated to provide a dosage form affording the advantage of prolonged action or to protect from the acid conditions of the stomach. The tablets may also be formulated for immediate release. In certain embodiments, the tablet comprises a film coating. A film coating may be useful for limiting photolytic degradation. Suitable film coatings are selected by routine screening of commercially available preparations. In one embodiment, the film coating is a hypromellose-based coating. In certain embodiments, the coating comprises a film-forming agent, a plasticizer, a glidant and optionally one or more pigments. An exemplary film coating composition may comprise hydroxypropyl methylcellulose (HPMC), lactose monohydrate, titanium dioxide, and triglyceride 1,2,3-triacetoxypropane (triacetin). In certain cases, the film coating composition may comprise hydroxypropyl methylcellulose (HPMC), polyethylene glycol (PEG), talc, titanium dioxide and optionally iron oxide, including iron oxide red and/or yellow.

In some embodiments, the pharmaceutical dosage form comprises a compound or amorphous solid dispersion (e.g., as described herein) in a therapeutically effective amount.

In some embodiments, the pharmaceutical dosage form includes a subject amorphous solid dispersion (e.g., as described herein) comprising TQS-168 in an amount from 10-5000 mg. In some embodiments, the pharmaceutical dosage form comprises 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg+/−10% of TQS-168. In certain embodiments, the pharmaceutical dosage form comprises 1500 mg, 2000 mg, 2500 mg, 3000 mg, 3500 mg, 4000 mg, 4500 mg, or 5000 mg+/−10% of TQS-168.

In some embodiments, the pharmaceutical dosage form includes a subject amorphous solid dispersion (e.g., as described herein) comprising TQS-168 in an amount of 25-2000 mg. In some embodiments, the pharmaceutical dosage form comprises 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg of TQS-168.

In some embodiments, the pharmaceutical dosage form includes a subject amorphous solid dispersion (e.g., as described herein) comprising TQS-168 in an amount of 50 mg to 450 mg. In some embodiments, the pharmaceutical dosage form comprises from 50 mg to 100 mg TQS-168, or from 100 mg to 150 mg TQS-168, or from 150 mg to 200 mg TQS-168, or from 250 mg to 300 mg TQS-168, or from 300 mg to 350 mg TQS-168, from 350 mg to 400 mg, or 400 mg to 450 mg of TQS-168. In some embodiments, the pharmaceutical dosage form comprises 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg+/−10% of TQS-168. In some embodiments, the pharmaceutical dosage form comprises 210 mg, 220 mg, 230, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, or 450 mg+/−10% of TQS-168. In an embodiment, the pharmaceutical dosage form comprises 50 mg+/−10% of TQS-168. In an embodiment, the pharmaceutical dosage form comprises 100 mg+/−10% of TQS-168. In an embodiment, the pharmaceutical dosage form comprises 150 mg+/−10% of TQS-168. In some embodiments, the pharmaceutical dosage form comprises 200 mg+/−10% of TQS-168. In some embodiments, the pharmaceutical dosage form comprises 300 mg+/−10% of TQS-168. In some embodiments, the pharmaceutical dosage form comprises 400 mg+/−10% of TQS-168. In some embodiments, the pharmaceutical dosage form comprises 450 mg+/−10% of TQS-168.

In some embodiments, the pharmaceutical dosage form includes a subject compound of formula (I) (e.g., as described herein) in an amount from 10-5000 mg. In some embodiments, the pharmaceutical dosage form comprises 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg+/−10% of a compound of formula (I). In certain embodiments, the pharmaceutical dosage form comprises 1500 mg, 2000 mg, 2500 mg, 3000 mg, 3500 mg, 4000 mg, 4500 mg, or 5000 mg+/−10% of a compound of formula (I).

In certain embodiments, the pharmaceutical dosage form comprises one or more excipients (e.g., as described herein). In certain embodiments, the pharmaceutical dosage form comprises one or more diluents. In certain embodiments, the pharmaceutical dosage from comprises microcrystalline cellulose.

In certain embodiments, the pharmaceutical dosage form comprises one or more diluents (e.g., microcrystalline cellulose) in an amount from 300 mg to 700 mg, such as 350 mg to 650 mg, 400 mg to 650 mg, 450 mg to 650 mg, or 500 to 600 mg. In some embodiment, the diluent is present in an amount of 500 mg, 510 mg, 520 mg, 530 mg, 540 mg, 550 mg, 560 mg, 570 mg, 580 mg, 590 mg, or 600 mg+/−10%.

In certain embodiments, the pharmaceutical dosage form comprises microcrystalline cellulose in an amount from 300 mg to 700 mg, such as 350 mg to 650 mg, 400 mg to 650 mg, 450 mg to 650 mg, or 500 to 600 mg. In a specific embodiment, the microcrystalline cellulose is in an amount of 500 mg, 510 mg, 520 mg, 530 mg, 540 mg, 550 mg, 560 mg, 570 mg, 580 mg, 590 mg, or 600 mg+/−10%.

In certain embodiments, the pharmaceutical dosage form comprises one or more disintegrants. In certain cases, the disintegrant is croscarmellose sodium. In certain embodiments, the pharmaceutical dosage form comprises a disintegrant in an amount from 15 mg to 65 mg, such as about 15 mg to 60 mg, 20 mg to 60 mg, 25 mg to 60 mg, 30 mg to 60 mg, 35 to 55 mg, or about 40 to 50 mg. In certain embodiments, the pharmaceutical dosage from comprises a disintegrant in an amount of from 45 mg to 55 mg, such as 45 mg, 46 mg, 47 mg, 48 mg, 49 mg, 50 mg, 51 mg, 52 mg, 53 mg, 54 mg, or 55 mg+/−10%. In some embodiments, the disintegrant is in an amount of 50 mg.

In certain embodiments, the pharmaceutical dosage form comprises croscarmellose sodium in an amount from 15 mg to 65 mg, such as about 15 mg to 60 mg, 20 mg to 60 mg, 25 mg to 60 mg, 30 mg to 60 mg, 35 to 55 mg, or about 40 to 50 mg. In certain embodiments, the pharmaceutical dosage from comprises croscarmellose sodium in an amount of from 45 mg to 55 mg, such as 45 mg, 46 mg, 47 mg, 48 mg, 49 mg, 50 mg, 51 mg, 52 mg, 53 mg, 54 mg, or 55 mg+/−10%. In an embodiment, the croscarmellose sodium is in an amount of 50 mg.

In certain embodiments, the pharmaceutical dosage form comprises one or more lubricants. In certain cases, the lubricant is sodium stearyl fumarate. In certain embodiments, the pharmaceutical dosage form comprises a lubricant in an amount from 1 mg to 15 mg, such as 2 to 12 mg, 2 to 10 mg, 3 to 10 mg, 4 to 10 mg, or 5 to 10 mg. In certain embodiments, the pharmaceutical dosage from comprises a lubricant in an amount from 5 mg to 10 mg, such as 5 mg, 6 mg, 7 mg, 8 mg, 9 mg, or 10 mg+/1 10%. In a specific embodiment, the lubricant is present in an amount of 10 mg.

In certain embodiments, the pharmaceutical dosage form comprises sodium stearyl fumarate in an amount from 1 mg to 15 mg, such as 2 to 12 mg, 2 to 10 mg, 3 to 10 mg, 4 to 10 mg, or 5 to 10 mg. In certain embodiments, the pharmaceutical dosage from comprises sodium stearyl fumarate in an amount from 5 mg to 10 mg, such as 5 mg, 6 mg, 7 mg, 8 mg, 9 mg, or 10 mg+/1 10%. In a specific embodiment, the sodium stearyl fumarate is in an amount of 10 mg.

In some embodiments, the pharmaceutical dosage form comprises one or more glidants. In some cases, the glidant is colloidal silicon dioxide. In some embodiments, the pharmaceutical dosage form comprises a glidant in an amount from 5 mg to 25 mg, such as about 10 to 25 mg, 10 to 20 mg, or 15 to 20 mg. In some embodiments, the pharmaceutical dosage from comprises a glidant in an amount from 15 to 20 mg, such as 15 mg, 16 mg, 17 mg, 18 mg, 19 mg, or 20 mg+/−10%. In an embodiment, the glidant is present in an amount of 15 mg. In an embodiment, the glidant is present in an amount of 20 mg.

In some embodiments, the pharmaceutical dosage form comprises colloidal silicon dioxide in an amount from 5 mg to 25 mg, such as about 10 to 25 mg, 10 to 20 mg, or 15 to 20 mg. In some embodiments, the pharmaceutical dosage form comprises colloidal silicon dioxide in an amount from 15 to 20 mg, such as 15 mg, 16 mg, 17 mg, 18 mg, 19 mg, or 20 mg+/−10%. In an embodiment, the colloidal silicon dioxide is present in an amount of 15 mg. In an embodiment, the colloidal silicon dioxide is present in an amount of 20 mg.

In some embodiments, the pharmaceutical dosage form comprises one or more sweeteners. In some cases, the sweetener is sucralose. In some embodiments, the pharmaceutical dosage form comprises a sweetener in an amount from 1 mg to 30 mg, such as about 1 to 25 mg, 1 to 23 mg, 5 to 25 mg, 10 to 25 mg, 15 to 25 mg, or 20 to 25 mg. In some embodiments, the pharmaceutical dosage from comprises a sweetener in an amount from 20 to 25 mg, such as 20 mg, 21 mg, 22 mg, 23 mg, 24 mg, or 25 mg+/−10%. In an embodiment, the sweetener is present in an amount of 22-23 mg.

In some embodiments, the pharmaceutical dosage form comprises sucralose in an amount from 1 mg to 30 mg, such as about 1 to 25 mg, 1 to 23 mg, 5 to 25 mg, 10 to 25 mg, 15 to 25 mg, or 20 to 25 mg. In some embodiments, the pharmaceutical dosage form comprises sucralose in an amount from 20 to 25 mg, such as 20 mg, 21 mg, 22 mg, 23 mg, 24 mg, or 25 mg+/−10%. In an embodiment, the colloidal silicon dioxide is present in an amount of 15 mg. In an embodiment, the sucralose is present in an amount of 22-23 mg.

In some embodiments, the pharmaceutical dosage form comprises a) 50-400 mg of an amorphous solid dispersion of TQS-168 (e.g., as described herein); b) 300-700 mg of a diluent; c) 15-65 mg of a disintegrant; d) 1-15 mg of a lubricant; e) 5-25 mg of a glidant; and f) optionally 1-25 mg of a sweetener.

In some embodiments, the pharmaceutical dosage form comprises a) 50-400 mg of an amorphous solid dispersion of TQS-168 (e.g., as described herein); b) 300-700 mg of microcrystalline cellulose; c) 15-65 mg of croscarmellose sodium; d) 1-15 mg of sodium stearyl fumarate; e) 5-25 mg of colloidal silicon dioxide; and f) optionally 1-25 mg of sucralose.

In some embodiments, the pharmaceutical dosage form includes an amorphous solid dispersion of TQS-168 (e.g., as described herein) as the sole active ingredient. In some embodiments, the pharmaceutical dosage form includes an amorphous solid dispersion of TQS-168 (e.g., as described herein) in combination with other active agents (e.g., as described herein).

In some embodiments, the subject pharmaceutical dosage form (e.g., as described herein) may be administered orally.

In some embodiments, the pharmaceutical dosage from comprises 2-(4-tert-butylphenyl)-1H-benzo[d]imidazol-5-ol (Compound 26) having the formula:

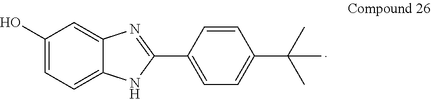 [SMILES: CC(C)(C)c1ccc(-c2nc3cc(O)ccc3[nH]2)cc1]

In some embodiments, the pharmaceutical dosage form includes a compound of formula (I) as the sole active ingredient. In some embodiments, the pharmaceutical dosage form includes a compound of formula (I) in combination with other active agents (e.g., as described herein).

In some embodiments, the pharmaceutical composition, or pharmaceutical dosage form as described herein is free of negative drug-drug interactions. In a related embodiment, the pharmaceutical composition, or pharmaceutical dosage form is free of negative drug-drug interactions with other active agents. In a further embodiment, the pharmaceutical composition, or pharmaceutical dosage form as described herein is administrable without regard to food and with or without regard to the patient being on another therapeutic agent.

### 3.5. Dosage Regimens

### 3.5.1. Unit Dose

In some embodiments, the compound of formula (I) or TQS-168 in the subject formulations is administered in a dose that is independent of subject weight or surface area (flat dose).

In some embodiments, the flat dose is 0.1 mg, 0.2 mg, 0.3 mg, 0.4 mg, 0.5 mg, 0.6 mg, 0.7 mg, 0.8 mg, 0.9 mg, or 1 mg. In some embodiments, the flat dose is 1 mg, 2 mg, 3 mg, 4 mg, 5 mg, 6 mg, 7 mg, 8 mg, 9 mg, or 10 mg. In some embodiments, the flat dose is 11 mg, 12 mg, 13 mg, 14 mg, 15 mg, 16 mg, 17 mg, 18 mg, 19 mg, or 20 mg. In some embodiments, the flat dose is 25 mg, 30 mg, 40 mg, or 50 mg. In some embodiments, the flat dose is 60 mg, 70 mg, 80 mg, 90 mg, or 100 mg. In some embodiments, the flat dose is 200 mg, 300 mg, 400 mg, 500 mg, 600 mg, 700 mg, 800 mg, 900 mg, or 1000 mg.

In some embodiments, the flat dose is 0.1-1 mg, 1-10 mg, 10-15 mg, 15-20 mg, 20-30 mg, 30-40 mg, or 40-50 mg. In some embodiments, the flat dose is 1-50 mg, 50-100 mg, 100 mg-200 mg, 200 mg-300 mg, 300 mg-400 mg, 400 mg-500 mg, 500 mg-600 mg, 600 mg-700 mg, 700 mg-800 mg, 800 mg-900 mg, or 900 mg-1000 mg.

In some embodiments, the flat dose is 10-5000 mg. In some embodiments, the dose is 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the dose is 1500 mg, 2000 mg, 2500 mg, 3000 mg, 3500 mg, 4000 mg, 4500 mg, or 5000 mg.

In some embodiments, the flat dose is 25-2000 mg. In certain embodiments, the dose is 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg. In some embodiments, the flat dose is 150 mg.

In various embodiments, the compound of formula (I) (e.g., as described herein); or the TQS-168 in the subject formulations (e.g., as described herein), is administered using a weight-based dose.

In some embodiments, the subject compound is administered in an amount of at least 0.5 mg/kg. In certain embodiments, the subject compound is administered in an amount of at least 1 mg/kg. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg.

In various embodiments, the dose is at least 10 mg/kg. In certain embodiments, the dose is at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 150 mg/kg, at least 175 mg/kg, or at least 200 mg/kg.

In some embodiments, the dose is 0.5 mg/kg to 100 mg/kg per day. In some embodiments, the dose is 2 mg/kg to 100 mg/kg per day. In some embodiments, the dose is 25 mg/kg to 1000 mg/kg per day. In certain embodiments, the dose is 25 mg/kg.

### 3.5.2. Dose Regimen

The compound of formula (I) (e.g., as described herein) or the subject formulation comprising TQS-168 (e.g., as described herein) can be administered in a single dose or in multiple doses. An exemplary dosage form may be a powder, granules (e.g., powder or granules reconstituted in a food or beverage for oral administration), an oral suspension, or an oral disintegrating tablet, taken from one to six times daily. In certain embodiments, the pharmaceutical dosage form is administered once daily. In certain embodiments, the pharmaceutical dosage form is administered twice daily. In some embodiments, the pharmaceutical dosage form is administered three times daily. In some embodiments, the pharmaceutical dosage form is administered four times daily. In some embodiments, the pharmaceutical dosage form is administered five times daily. In some embodiments, the pharmaceutical dosage form is administered six times daily.

In some embodiments, multiple doses of the pharmaceutical dosage form are administered. The frequency of administration of the dosage form can vary depending on any of a variety of factors, e.g., severity of the symptoms, etc. For example, in some embodiments, the subject dosage form is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). In certain embodiments, the subject dosage form is administered twice a day (bid). In certain embodiments, the subject dosage form is administered three times a day (tid).

The duration of administration of a pharmaceutical dosage from, e.g., the period of time over which the subject compound is adrninistered, can vary, depending on any of a variety of factors, e.g., patient response, etc. For example, an active agent can be administered over a period of time ranging from about one day to about one week, from about two weeks to about four weeks, from about one month to about two months, or from about two months to about four mionths, or more,

Those of skill in the art will readily appreciate that dose levels can vary as a function of the specific compound, the severity of the symptoms and the susceptibility of the subject to side effects. Dosages for a given compound are readily determinable by those of skill in the art by a variety of means.

In some embodiments, the dosage form is administered intravenously. In one embodiment, the subject compound or formulation is administered once a day, once every 2 days, once every 3 days, once every 4 days, once every 5 days, once every 6 days, once every 7 days, once every 14 days, once every 21 days, once every 28 days, or once a month. In various intravenous embodiments, the subject compound o formulation is administered twice a day, twice every 2 days, twice every 3 days, twice every 4 days, twice every 5 days, twice every 6 days, twice every 7 days, twice every 14 days, twice every 21 days, twice every 28 days, or twice a month.

In various enteral embodiments, the subject compound or formulation is administered once a day, once every 2 days, once every 3 days, once every 4 days, once every 5 days, once every 6 days, once every 7 days, once every 14 days, once every 21 days, once every 28 days, or once a month. In various enteral embodiments, the compound of Formula I is administered twice a day, twice every 2 days, twice every 3 days, twice every 4 days, twice every 5 days, twice every 6 days, twice every 7 days, twice every 14 days, twice every 21 days, twice every 28 days, or twice a month.

In some embodiments, the subject compound is administered at a daily oral dose of 100-1000 mg. In some embodiments, the compound is administered at a daily oral dose of 200-800 mg. In some embodiments, the compound is administered at a daily oral dose of 300-700 mg. In some embodiments, the compound is administered at a daily oral dose of 300-600 mg. In some embodiments, the compound is administered at a daily oral dose of 400-600 mg. In some embodiments, the compound is administered at a daily oral dose of 400-500 mg.

In various embodiments, the subject compound is administered at a daily oral dose of 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the compound is administered at a daily oral dose of 100 mg. In certain embodiments, the compound is administered at a daily oral dose of 200 mg. In certain embodiments, the compound is administered at a daily oral dose of 400 mg. In certain embodiments, the compound is administered at a daily oral dose of 450 mg. In certain embodiments, the compound is administered at a daily oral dose of 500 mg. In certain embodiments, the compound is administered at a daily oral dose of 750 mg. In certain embodiments, the compound is administered at a daily oral dose of 1000 mg.

In some embodiments, the pharmaceutical dosage form comprises 100-200 mg the subject compound, and is administered orally 1-3 times daily. In certain embodiments, the dosage form is administered 3 times daily.

In some embodiments, the pharmaceutical dosage form comprises 200-300 mg the subject compound, and is administered orally 1-3 times daily. In certain embodiments, the dosage form is administered 2 times daily.

In some embodiments, the pharmaceutical dosage form comprises 300-400 mg the subject compound, and is administered orally 1-3 times daily. In certain embodiments, the dosage form is administered 2 times daily.

In some embodiments, the pharmaceutical dosage form comprises 400-500 mg the subject compound, and is administered orally 1-3 times daily. In certain embodiments, the dosage form is administered once daily.

### 3.6. Additional Agents

In some embodiments, the methods of the present disclosure further comprise administering an effective amount of at least one additional active agent. In certain embodiments, the additional active agent is selected from an agent approved for the treatment of a hemoglobinopathy. In some cases, the agent is approved for the treatment of a sickle cell disease. In certain embodiments, the one or more additional agents is selected from hydroxyurea, L-glutamine, crizanlizumab and voxelotor, or pharmacutically acceptable salts there of.

In some embodiments, the additional active agent is hydroxyurea.

In some embodiments, the additional active agent is L-glutamine.

In some embodiments, the additional active agent is crizanlizumab.

In some embodiments, the additional active agent is voxelotor.

In some embodiments of the subject methods, the compound of formula (I) (e.g., as described herein) or the formulation of TQS-168 (e.g., as described herein) in combination with one or more additional active agents (e.g., as described herein) has an additive effect on HbF production.

In some embodiments of the subject methods, the compound of formula (I) (e.g., as described herein) or the formulation of TQS-168 (e.g., as described herein) in combination with one or more additional active agents (e.g., as described herein) has a synergistic effect on HbF production.

In some embodiments of the subject methods, the compound of formula (I) is administered in combination with one or more additional agents selected from hydroxyurea, L-glutamine, crizanlizumab and voxelotor.

In some embodiments of the subject methods, the pharmaceutical composition comprising an amorphous solid dispersion of TQS-168 is administered in combination with one or more additional agents selected from hydroxyurea, L-glutamine, crizanlizumab and voxelotor.

In some embodiments of the subject methods, the compound of formula (I) is administered in combination with hydroxyurea.

In some embodiments of the subject methods, the pharmaceutical composition comprising an amorphous solid dispersion of TQS-168 is administered in combination with hydroxyurea.

In some embodiments of the subject methods, the compound of formula (I) is administered in combination with L-glutamine.

In some embodiments of the subject methods, the pharmaceutical composition comprising an amorphous solid dispersion of TQS-168 is administered in combination with L-glutamine.

In some embodiments of the subject methods, the compound of formula (I) is administered in combination with crizanlizumab.

In some embodiments of the subject methods, the pharmaceutical composition comprising an amorphous solid dispersion of TQS-168 is administered in combination with crizanlizumab.

In some embodiments of the subject methods, the compound of formula (I) is administered in combination with voxelotor.

In some embodiments of the subject methods, the pharmaceutical composition comprising an amorphous solid dispersion of TQS-168 is administered in combination with voxelotor.

### 3.7. Definitions

Unless defined otherwise, all technical and scientific terms used herein have the same meaning as is commonly understood by one of ordinary skill in the art to which this disclosure belongs. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations”, is incorporated herein by reference. In the event that there is a plurality of definitions for terms cited herein, those in this section prevail unless otherwise stated.

As used herein, the term “solid dispersion” is meant a molecular dispersion of a compound, particularly a drug substance within a carrier. The term solid dispersion in general means a system in solid state comprising at least two components, wherein one component is dispersed substantially evenly throughout the other component(s). For example, solid dispersions may be the dispersion of one or more active ingredients in an inert carrier or matrix at solid state, prepared by the melting, solvent, or melting-solvent methods. While not wishing to be bound by theory, ii a solid dispersion, the drug may be present in a molecular state, colloidal state, metastable state, or an amorphous state. Formation of a molecular dispersion may provide a means of reducing the particle size to nearly molecular levels (i.e., there are no particles).

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle etc. with which the compound is administered, without limitation. Examples of carriers are described herein and also in Remington: The Science and Practice of Pharmacy (Remington: The Science and Practice of Pharmacy, 23rd Edition, ISBN-13: 978-0128200070).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropyl cellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, modified corn starch, sodium carboxymethyl starch, crospovidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The term “film coating” refers to a thin, uniform, film on the surface of a substrate (e.g., tablet). Film coatings are particularly useful for protecting the active ingredient from photolytic degradation. Non-limiting examples of film coatings include polyvinylalcohol based, hydroxyethyl cellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient (e.g., a sachet or a tablet).

The term “treatment” or “treating,” to the extent it relates to a disease or condition includes preventing the disease or condition from occurring, inhibiting the disease or condition, eliminating the disease or condition, and/or relieving one or more symptoms of the disease or condition. Treatment can involve administering a compound described herein to a subject diagnosed with a disease, and may involve administering the compound to a subject who does not have active symptoms. Conversely, treatment may involve administering the compositions to a subject at risk of developing a particular disease, or to a subject reporting one or more of the physiological symptoms of a disease, even though a diagnosis of this disease may not have been made.

The terms “administer”, “administering” or “administration” in reference to a dosage form of the disclosure refers to the act of introducing the dosage form into the system of subject in need of treatment. When a dosage form of the disclosure is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

C₁ to C₂₀ hydrocarbon includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbyl refers to any substituent comprised of hydrogen and carbon as the only elemental constituents. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified, alkyl (or alkylene) is intended to include linear or branched saturated hydrocarbon structures and combinations thereof. Alkyl refers to alkyl groups from 1 to 20 carbon atoms, such as 1 to 10 carbon atoms, or 1 to 6 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Heterocycle means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Alkoxy or alkoxyl refers to groups of from 1 to 20 carbon atoms, such as 1 to 10 carbon atoms, or 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

The term “halogen” means fluorine, chlorine, bromine or iodine atoms. In one embodiment, halogen may be a fluorine or chlorine atom.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons. The double bonded oxygen, when referred to as a substituent itself is called “oxo”.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino[HNC(═O)O-alkyl], aminocarbonyl (also known as carboxamido) [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g., on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine.

Substituents Rⁿ are generally defined when introduced and retain that definition throughout the specification and in all independent claims. For any and all compounds shown or claimed, wherein tautomerism is possible, all possible tautomers are intended to be included.

The term “% w/w” as used herein refers to the weight of a component based on the total weight of a composition comprising the component. For example, if component A is present in an amount of 50% w/w in a 100 mg composition, component A is present in an amount of 50 mg.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### 4. EXAMPLES

The Examples in this section are offered by way of illustration, and not by way of limitation. The examples can represent only some embodiments, and it should be understood that the following examples are illustrative and not limiting. All excipients, unless otherwise specified, are as previously defined. The excipients and starting materials are readily available to one of ordinary skill in the art. The specific steps for each of the routes described may be combined in different ways, or in conjunction with steps from different processes, to prepare the formulations described herein.

### 4.1. Example 1: Synthesis of Compounds of Formula (I)

Compounds of formula (I) may be synthesized using any convenient method. Methods which can be adapted for use in preparing compounds of this disclosure includes the exemplary synthetic methods described in Methods A-H of Banister et al. in PCT application No. PCT/US2019/045229, filed Aug. 6, 2019, the disclosure of which is herein incorporated by reference in its entirety. Reactions may be monitored by thin layer chromatography (TLC), LC/MS and reaction products characterized by LC/MS and 1H NMR. Intermediates and final products may be purified by silica gel chromatography or by HPLC.

### 4.2. Example 2: Preparation of a Spray Dried Dispersion (SDD) Formulation of 2-(4-tert-butylphenyl)-1H-benzimidazole (TQS-168)

A spray-dried dispersion (SDD) of 2-(4-tert-butylphenyl)-1H-benzimidazole (TQS-168) having the composition set out in Table 4 was prepared by spray drying a feedstock formulation set out in Table 5.

TABLE 4
Spray Dried Dispersion (SDD) Formulation of TQS-168

| | % | Amount |
|---|---|---|
| Ingredient | w/w | (g) |
| TQS-168 | 30 | 45.0 |
| polyvinyl caprolactam-polyvinyl | 40 | 60.0 |
| acetate-polyethylene glycol graft | | |
| copolymer (Soluplus) | | |
| amorphous silicon dioxide (Syloid ® 244 FP) | 30 | 45.0 |
| Total | 100 | 150 |

TABLE 5
Spray Drying Feedstock Formulation

| | % | Amount |
|---|---|---|
| Ingredient | w/w | (g) |
| TQS-168 | 2.3 | 45.0 |
| polyvinyl caprolactam-polyvinyl | 3.1 | 60.0 |
| acetate-polyethylenc glycol graft | | |
| copolymer (Soluplus) | | |
| amorphous silicon dioxide (Syloid ® 244 FP) | 2.3 | 45.0 |
| 2-propanol | 92.3 | 1797.1 |
| Total | 100 | 1947.1 |

### Manufacturing Procedure for Spray Drying Feedstock Formulation

TQS-168 (45.0 g) was slowly added to 2-propanol (1791.1 g) with stirring, placed under a homogenizer (Silverson SL2 homogenizer) and stirred for 5 minutes or more until TQS-168 was fully dissolved. The reaction mixture was then removed from the homogenizer and polyvinyl caprolactam-polyvinyl acetate-polyethylene glycol graft co-polymer (Soluplus) (60.0 g) was slowly added with stirring, placed back under the homogenizer and stirred for 10 minutes or more until the Soluplus was fully dissolved. The reaction mixture was then removed from the homogenizer and amorphous silicon dioxide (Syloid® 244 FP) was slowly added with stirring, placed back under the homogenizer and stirred for an additional 15 minutes or more until the amorphous silicon dioxide was fully dispersed. The resulting suspension is referred to herein as the “Feedstock Formulation.”

### Manufacturing Procedure for Spray Dried Dispersion (SDD) Formulation of TQS-168

The spray dryer unit (ProCepT 4M8 Spray Dryer) was set up with a compressed air supply. Once the outlet temperature stabilized, the feed pump was initiated, and 2-propanol (blank solution) was sprayed through the nozzle as a fine spray into the collection chamber. The spray dryer parameters were adjusted to achieve a feed rate within the range set out in Table 6 below.

The feedstock formulation was stirred under a homogenizer at a speed appropriate to maintain a homogenous dispersion without generating bubbles. The feedstock formulation was then sprayed through the nozzle as a fine spray into the collection chamber of the spray dryer unit (ProCepT 4M8 Spray Dryer, using parameters as set up with the blank solution and outlined in Table 4) where the solvent was evaporated quickly to generate particles containing TQS-168 polyvinyl caprolactam-polyvinyl acetate-polyethylene glycol graft co-polymer (Soluplus) and silicon dioxide (Syloid® 244 FP) (SDD formulation of TQS-168). Once all the feedstock formulation had been sprayed and collected, the feedstock formulation was replaced with 2-propanol (blank solution) and sprayed through the nozzle of the spray dryer for 5 minutes or more to allow collection of any remaining “feedstock formulation” within the air stream.

TABLE 6
Target Process Parameters for Spray Dryer

| Process Parameter | Target | |
|---|---|---|
| Inlet air flow | 0.45 | m³/min |
| Inlet air temperature | 88° | C. |
| Suspension feed rate | 4-7 | g/min |
| Peristaltic tubing | 50513 Tygon MHLL ID 2.79 mm | |
| | (2-stop, purple-white) | |
| Nozzle diameter | 0.6 | mm |
| Nozzle cap | with cap | |
| Nozzle pressure | 2 | bar |
| Cyclone gas pressure | 0.3 | bar |

### 4.3. Example 3: Oral Suspension of a Spray Dried Dispersion (SDD) Formulation of 2-(4-tert-butylphenyl)-H-benzimidazole (TQS-168)

The spray-dried dispersion (SDD) of 2-(4-tert-butylphenyl)-1H-benzimidazole (TQS-168) having the composition set out in Table 4 (e.g., from 60-1000 mg) was reconstituted as an oral suspension in 100 g of vehicle composed of PEG 300 (10 g), glycerol monocaprylocaprate (Capmul MCM, 0.40 mg) in sterile water for irrigation (q.s. to 100 g).

### 4.4. Example 4: Preparation of a Hot-Melt Extrusion (HME) Formulation of 2-(4-tert-butylphenyl)-1H-benzimidazole (TQS-168)

A hot-melt extrusion (HME) formulation of 2-(4-tert-butylphenyl)-IH-benzimidazole (TQS-168) having the composition set out in Table 7 was prepared as set out below.

TABLE 7
Hot-Melt Extrusion (HME) Formulation of TQS-168

| | | Amount | Amount |
|---|---|---|---|
| | | (mg) for | (mg) for |
| | | 60 mg | 1000 mg |
| Ingredient | % w/w | dose | dose |
| TQS-168 | 25 | 60 | 1000 |
| Povidone (Kollidon 17PF) | 25 | 60 | 1000 |
| Copovidone (Kollidon VA64) | 25 | 60 | 1000 |
| polyvinyl caprolactam-polyvinyl | 25 | 60 | 1000 |
| acetate-polyethylene | | | |
| glycol graft co-polymer (Soluplus) | | | |
| Total | 100 | 240 | 4000 |

### Manufacturing Procedure

The required quantities of TQS-168, polyvinyl caprolactam-polyvinyl acetate-polyethylene glycol graft co-polymer (Soluplus), povidone (Kollidon 17PF) and copovidone (Kollidon VA64) according to table 7 were weighed, passed through an 850 n sieve and transferred to a 2L blender shell. The resultant blender shell was secured in a blender (Pharmatech blender) and blended for 20 minutes then added to a double polyethylene (PE) bag (“TQS-168 blend”) and transferred to the HME containment system. A chiller unit was connected to the HME and once the chiller temperature reached 15° C. the extrusion process was commenced using the parameters outlined in Table 8. The TQS-168 blend was added to the feeder to fill approximately ¾ of the feeder, and the extrudate was collected and discarded for approximately the first 5 minutes of the extrusion process. The feeder was refilled to maintain approximately 50% volume in the feeder throughout the process, and extrusion was continued until all the TQS-168 blend was extruded and collected (“TQS-168 HME extrudate”).

TABLE 8
HME parameters

| Feed rate | 0.110-0.175 kg/hour | | | | | | | |
|---|---|---|---|---|---|---|---|---|
| Temperature (all | Zone 2 | Zone 3 | Zone 4 | Zone 5 | Zone 6 | Zone 7 | Zone 8 | Die |
| zones) | 50-70° C. | 80-170° C. | 200-220° C. | 220-240° C. | 220-240° C. | 220-240° C. | 220-240° C. | 170-195° C |

| | |
|---|---|
| Screw speed | 30 rpm |
| Die Dimension | 1.0 mm |
| Chiller | 15° C. |
| temperature | |

The collected TQS-168 HME extrudate was added to a U5 Quadro mill (set up with a screen size of 457 (mm) and an impeller speed of 5000 RPM), until all extrudate had passed the 457 mm screen to obtain milled granules of TQS-168. The milled granules of TQS-168 were then sieved using a 300 micron sieve and transferred into a blender shell (Pharmatech 2L blender shell). The resultant blender shell was secured in a blender (Pharmatech blender), blended for 5 minutes, and collected.

### 4.5. Example 5: Oral Suspension of a Hot-Melt Extrusion (HME) Formulation of 2-(4-tert-butylphenyl)-1H-benzimidazole (TQS-168)

The hot-melt extrusion (HME) of 2-(4-tert-butylphenyl)-1H-benzimidazole (TQS-168) having the composition set out in Table 7 (e.g., from 60-1000 mg) was reconstituted as an oral suspension in 100 mL of vehicle Ora-Blend SF® (purified water, sucrose, glycerin, sorbitol, flavoring, microcrystalline cellulose, sodium carboxymethylcellulose, xanthan gum, carrageenan, citric acid, sodium phosphate, simethicone, potassium sorbate and methylparaben), a commercially available oral suspending vehicle manufactured by Perrigo Pharmaceuticals.

### 4.6. Example 6: Preparation of a Spray Dried Dispersion (SDD) Blend Formulation of 2-(4-tert-butylphenyl)-1H-benzimidazole (TQS-168)

### General Manufacturing Procedure for SDD Blend Formulations

The required amount of TQS-168 SDD formulation (e.g., as prepared in accordance with Example 2) was weighed, sieved through a 250 m sieve and transferred to a suitable blending shell. The required amount of diluent was then weighed, sieved through a 250 μm sieve and transferred to the suitable blending shell with the TQS-168 SDD formulation (“the blending shell”). The resultant mixture was blended for 3 minutes at 25 rpm (Pharmatech blender). The disintegrant, sweetener and lubricant were then weighed, sieved through a 250 m sieve and transferred to the blending shell. The resultant mixture was blended at 25 rpm (Pharmatech blender) for approximately 60 minutes. The SDD blends of TQS-168 were then evaluated for bulk density, tapped density, Carr's Index and Hausner ratio (see Tables 11-13 below) before filling into sachets. Sachets of each blend (10 sachets of each) were then submitted for content uniformity testing (see Table 14 below).

The TQS-168 SDD blend formulation compositions are summarized in Tables 9-10.

TABLE 9
TQS-168 SDD blend formulation compositions 1-3

| | | | 1 + 2% | | | | |
|---|---|---|---|---|---|---|---|
| | | | colloidal | | | | |
| Batch No. | 1 | | silica | 2 | | 3 | |
| Excipients | mg/sachet | % w/w | mg/sachet | mg/sachet | % w/w | mg/sachet | % w/w |
| TQS-168 SDD | 400.0 | 40.0 | 400.0 | 400.0 | 40.0 | 400.0 | 40.0 |
| Microcrystalline | 588.5 | 58.85 | 588.5 | | | | |
| cellulose | | | | | | | |
| (particle size = | | | | | | | |
| 100 μm) | | | | | | | |
| Partially | | | | 588.5 | 58.85 | | |
| pregelatinized | | | | | | | |
| starch (Starch | | | | | | | |
| 1500) | | | | | | | |
| Mannitol (100 | | | | | | 588.5 | 58.85 |
| SD) | | | | | | | |
| Sodium stearyl | 10.0 | 1.00 | | | | | |
| fumarate | | | | | | | |
| Sucralose | 1.5 | 0.15 | 1.5 | 1.5 | 0.15 | 1.5 | 0.15 |
| Colloidal silica | | | 20.0 | | | | |
| (specific surface | | | | | | | |
| area = 200 | | | | | | | |
| m²/g) | | | | | | | |
| Total | 1000 | 100 | 1020 | 1000 | 100 | 1000 | 100 |

TABLE 10
TQS-168 SDD blend formulation compositions 4-5

| Batch No. | 4 | | 5 | |
|---|---|---|---|---|
| Excipients | mg/sachet | % w/w | mg/sachet | % w/w |
| TQS-168 SDD | 400.0 | 40.0 | 400.0 | 40.0 |
| Microcrystalline cellulose | | | 502.50 | 50.25 |
| (particle size = 50 μm) | | | | |
| Silicified microcrystalline | 567.50 | 56.75 | | |
| cellulose (SMCC HD 90) | | | | |
| Croscarmellose sodium | | | 50.0 | 5.00 |
| (Ac-Di-Sol) | | | | |
| Sodium stearyl fumarate | 10.0 | 1.0 | 10.0 | 1.0 |
| Sucralose | 22.50 | 2.25 | 22.50 | 2.25 |
| Colloidal silica (specific | | | 15 | 1.50 |
| surface area = 200 m²/g) | | | | |
| Total | 1000 | 100 | 1000 | 100 |

The results for bulk density, tapped density, Carr's Index and Hausner ratio for the TQS-168 SDD blend formulation compositions are summarized in Tables 11 and 12.

TABLE 11
Flow properties for TQS-168 SDD blend formulation compositions 1-3

| Flow Properties | 1 | 1 + 2% colloidal silica | 2 | 3 |
|---|---|---|---|---|
| Bulk Density | 0.36 | 0.32 | 0.40 | 0.34 |
| (mg/mL) | | | | |
| Tapped Density | 0.57 | 0.52 | 0.63 | 0.56 |
| (mg/mL) | | | | |
| Carr Index | 36.36 | 38.24 | 36.67 | 40.00 |
| Hausner Ratio | 1.57 | 1.62 | 1.58 | 1.67 |

TABLE 12
Flow properties for TQS-168 SDD blend formulation
compositions 4-5 and TQS-168 SDD

| Flow Properties | TQS-168 SDD | 4 | 5 |
|---|---|---|---|
| Bulk Density (mg/mL) | 0.36 | 0.36 | 0.33 |
| Tapped Density (mg/mL) | 0.57 | 0.53 | 0.52 |
| Carr Index % | 36.36 | 32 | 37 |
| Hausner Ratio | 1.57 | 1.47 | 1.58 |

Table 13 shows the relationship between Carr Index (compressibility index) and powder flow properties.

TABLE 13
Relationship between Carr Index and powder flow properties

| Carr Index (%) | Flow Character | Hausner Ratio |
|---|---|---|
| ≤10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very Poor | 1.46-1.59 |
| ≥38 | Very, Very Poor | ≥1.6 |

Table 14 shows the results of content uniformity testing for TQS-168 SDD blend formulations.

TABLE 14
Content Uniformity Results for TQS-168 SDD blend
formulation compositions 1-5

| Blend | Mean % Content Uniformity (CU) |
|---|---|
| 1 | 92.6 |
| 2 | 91.0 |
| 3 | 90.9 |
| 4 | 91.2 |
| 5 | 94.3 |

### 4.7. Example 7: Effects of Subject Compounds and Formulations on Human Primary Erythroid Progenitor CD34⁺ Cells

The effects of the compounds and formulations described in Sections 3.2 and 3.3 hereinabove on human primary erythroid progenitor CD34⁺ cells are studies using methods adapted from Sun et al. Sun et al., Br J Haematol. 197:97-109 (2022), the disclosure of which is herein incorporated by reference in its entirety. HbF-positive cells (F cells) are quantified by flow cytometry after compound exposure. Globin gene mRNA abundance is determined by quantitative real-time polymerase chain reaction (qRT-PCR).

The tested compounds and formulations, including 2-(4-tert-butylphenyl)-1H-benzo[d]imidazol-5-ol (Compound 26) and TQS-168, induce both fetal γ-globin mRNA protein expression and percentage of HbF-positive cells (F cells) in human primary erythroid progenitor CD34⁺ cells.

### 4.8. Example 8: Effects of Subject Compounds and Formulations on SCD Mice

The effects of the compounds and formulations described in Sections 3.2 and 3.3 on SCD mice are studied using methods adapted from Sun et al., Br J Haematol. 197:97-109 (2022), the disclosure of which is herein incorporated by reference in its entirety. Relative mRNA abundance of PGC-1α and globin gene expression are determined by quantitative real-time polymerase chain reaction (qRT-PC R). HbF-positive cells (F cells) in peripheral blood of treated SCD mice are quantified by flow cytometry

The tested compounds and formulations, including 2-(4-tert-butylphenyl)-1H-benzo[d]imidazol-5-ol (Compound 26) and TQS-168, induce expression of both the murine embryonic βh1-globin gene and the human γ-globin gene expression in SCD mice. The subject compounds/formulations also induce F-cell population in the peripheral blood of SCD mice.

### 5. EQUIVALENTS AND INCORPORATION BY REFERENCE

While the disclosure has been particularly shown and described with reference to a particular embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

1. A method of treating a hemoglobinopathy in a subject, comprising administering to a subject suffering from a hemoglobinopathy an effective amount of a compound of formula (I): 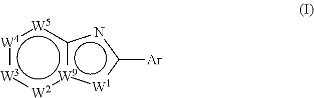
or a pharmaceutically acceptable salt, solvate, deuterated derivative, or prodrug thereof, wherein:
Ar is 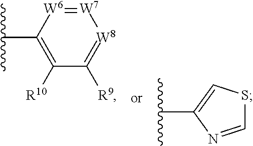
W¹ is chosen from O, S, and N—R¹, or, when W⁹ is N, W¹ may additionally be C—R⁵⁰;
W² is N or C—R²;
W³ is N or C—R³;
W⁴ is N or C—R⁴;
W⁵ is N or C—R⁵;
W⁶ is N or C—R⁶;
W⁷ is N or C—R⁷;
W⁸ is N or C—R⁸;
W⁹ is C, or, when W¹ is C—R⁵⁰; W⁹ may be N;
wherein:
R¹ is chosen from H, (C₁-C₃)alkyl, —CH₂OC(═O)R³⁰; —CH₂OP(═O)OR⁴⁰OR⁴¹, —C(═O)OR⁴², and —C(═O)R⁴³;
wherein:
R³⁰ is chosen from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
wherein:
R⁴⁴ is chosen from any naturally occurring amino acid sidechain;
R⁴⁵ is chosen from H, methyl, and (C₁-C₄)alkoxycarbonyl;
R⁴⁰ and R⁴¹ are chosen independently from hydrogen and (C₁-C₆)hydrocarbyl;
R⁴² is (C₁-C₅)alkyl;
R⁴³ is (C₁-C₃)alkyl; and
R⁵⁰ is H or (C₁-C₃)alkyl;
R², R³, R⁴ and R⁵ are chosen independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are chosen independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are chosen independently from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy, 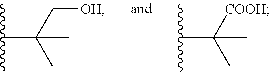
and
R⁸ is chosen from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino, 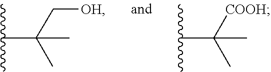
with the proviso that:
i) when Ar is 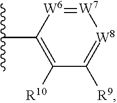
R⁸ is not (C₁-C₄)alkyl when:
(a) W¹ is N—R¹;
(b) R¹ is hydrogen;
(c) W², W³, W⁴, W⁵, W⁶, and W⁷ are C—H;
(d) W⁸ is C—R⁸;
(e) W⁹ is C; and
(f) R⁹ and R¹⁰ are hydrogen.

2. The method of claim 1, wherein the hemoglobinopathy is a β-hemoglobinopathy.

3. The method of claim 2, wherein the hemoglobinopathy is a sickle cell disease (SCD), a sickle cell trait (SCT) or a β-thalassemia.

4. The method of claim 3, wherein the hemoglobinopathy is SCD.

5. The method of claim 4, wherein the SCD is selected from sickle cell anemia (HbSS), sickle-hemoglobin C disease (HbSC), sickle beta-plus-thalassemia (HbS/β+), and sickle beta-zero-thalassemia (HbS/βO).

6. The method of claim 5, wherein the sickle cell disease is sickle cell anemia (HbSS).

7. The method of claim 6, wherein the subject is identified as suffering from a sickle cell crisis, or has been identified as being at risk of incurring a sickle cell crisis prior to treatment.

8. The method of claim 1, wherein the compound of formula (I) is of the formula (II): 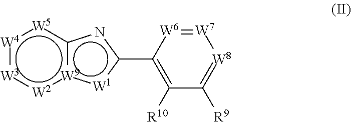
with the proviso that R⁸ is not hydrogen or (C₁-C₄)alkyl when:
(a) W¹ is N—R¹;
(b) R¹ is hydrogen;
(c) W², W³, W⁴, W⁵, W⁶, and W⁷ are C—H;
(d) W¹ is C—R⁸;
(e) W⁹ is C; and
(f) R⁹ and R¹⁰ are hydrogen.

9. The method of claim 8 wherein:
(a) W¹ is N—R¹ and R¹ is hydrogen;
(b) W² is C—R², wherein R² is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(c) W³ is C—R³, wherein R³ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(d) W⁴ is C—R⁴, wherein R⁴ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(e) W⁵ is C—R⁵, wherein R⁵ is selected from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
(f) W⁶ is C—R⁶, wherein R⁶ is selected from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
(g) W⁷ is C—R⁷, wherein R⁷ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy;
(h) W⁸ is C—R⁸, wherein R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino;
(i) W¹ is NR¹, O, or S and W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is selected from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino.

10. The method of claim 8 wherein:
(a) W¹ is N—R¹ and R¹ is hydrogen;
(b) W² is C—R², wherein R² is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(c) W³ is C—R³, wherein R³ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(d) W⁴ is C—R⁴, wherein R⁴ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(e) W⁵ is C—R⁵, wherein R⁵ is selected from hydrogen, perfluoro(C₁-C₄)alkyl, (C₁-C₄)alkyl, (C₁-C₄)alkoxy, perfluoro(C₁-C₄)alkoxy, amino, hydroxy, nitrile, halo and carboxamide;
(f) W⁶ is C—H;
(g) W⁷ is C—R⁷, wherein R⁷ is hydrogen or (C₃-C₄)alkyl;
(h) W¹ is C—R¹, wherein R¹ is selected from H, (C₁-C₄)alkyl, amino, (C₁-C₄)alkoxy, halo(C₁-C₄)alkoxy, and hydroxy;
(i) W¹ is NR¹; and W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is hydrogen.

11. The method of claim 8 wherein:
(a) W¹ is N—R¹ and R¹ is hydrogen;
(b) W² is selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
(c) W³ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br;
(d) W⁴ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br;
(e) W⁵ is selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
(f) W⁶ is C—H;
(g) W⁷ is C—R⁷, wherein R⁷ is hydrogen or (C₃-C₄)alkyl;
(h) W⁸ is C—R⁸, wherein R⁸ is selected from H, tert-butyl, amino, and methoxy;
(i) W⁹ is C;
(j) R⁹ is selected from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy; and
(k) R¹⁰ is hydrogen.

12. The method of claim 1, wherein the compound is of the formula (III): 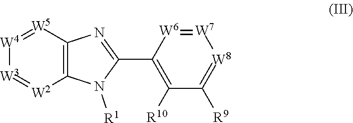
wherein:
R¹ is selected from —CH₂OC(═O)R³⁰, —CH₂OP(═O)OR⁴⁰OR⁴¹, —C(═O)OR⁴², and —C(═O)R⁴³;
wherein:
R³⁰ is selected from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
wherein:
R⁴⁴ is chosen from any naturally occurring amino acid sidechain;
R⁴⁵ is chosen from H, methyl, and (C₁-C₄)alkoxycarbonyl;
R⁴⁰ and R⁴¹ are chosen independently from hydrogen and (C₁-C₆)hydrocarbyl;
R⁴² is (C₁-C₅)alkyl; and
R⁴³ is (C₁-C₃)alkyl.

13. The method of claim 12, wherein:
W⁶ is C—H; W¹ is C—R⁸;
R⁹ and R¹⁰ are hydrogen;
R⁷ is H or (C₃-C₄)alkyl;
R⁸ is selected from H, (C₁-C₄)alkyl, amino, (C₁-C₄)alkoxy, halo(C₁-C₄)alkoxy, and hydroxy;
W² and W⁵ are C—R² and C—R⁵, respectively;
R², R³, R⁴, and R⁵ are independently selected from H, halo, and perfluoro(C₁-C₃)alkyl; and
R¹ is —CH₂OP(═O)OR⁴⁰OR⁴¹ or —CH₂OC(═O)R³⁰.

14. The method of claim 12, wherein:
W² and W⁵ are independently selected from C—H, C—F, C-D, C—CF₂H, C-CD₃, and C—CF₃;
W³ is selected from N, C—H, C—NH₂, C—F, C—CF₃, C—CF₂H, C-CD₃, and C-D;
W⁴ is selected from N, C—H, C—NH₂, C—F, C—CF₃, C—CF₂H, C-CD₃, and C-D;
W⁷ is C—H;
R⁸ is selected from H, tert-butyl, amino, and methoxy; and
R⁹ is H or tert-butyl.

15. The method of claim 12, wherein from one to four of W², W³, W⁴ and W⁵ are C-D or C—F.

16. The method of claim 12, wherein W², W³, W⁴ and W⁵ are C—R², C—R³, C—R⁴ and C—R⁵ respectively, and one of R², R³, R⁴ and R⁵ is perfluoro(C₁-C₄)alkyl, C₁-C₄)alkyl, (C₁-C₄)alkoxy, perfluoro(C₁-C₄)alkoxy, amino, hydroxy, nitrile, halo or carboxamide, and the remainder are hydrogen.

17. The method of claim 12, wherein one of W³ or W⁴ is N and the other, along with W² and W⁵, is C—H.

18. The method of claim 12, wherein W², W³, W⁴, W⁵, W⁶, and W⁷ are C—H.

19. The method of claim 12, wherein R⁸ is t-butyl.

20. The method of claim 1, wherein:
Ar is 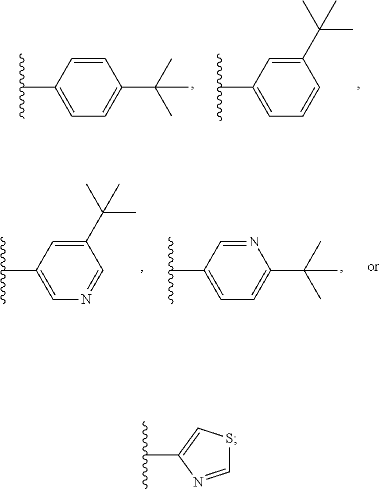
W¹ is selected from O, S, and N—R¹, or, when W⁹ is N, W¹ may additionally be C—R⁵⁰;
W² is N or C—R²;
W³ is N or C—R³;
W⁴ is N or C—R⁴;
W⁵ is N or C—R⁵;
W⁹ is C, or, when W¹ is C—R⁵⁰; W⁹ may be N;
wherein:
R¹ is selected from H, (C₁-C₃)alkyl —CH₂OC(═O)R³⁰; CH₂OP(═O)OR⁴⁰OR⁴¹, —C(═O)OR⁴², and —C(═O)R⁴³;
wherein:
R³⁰ is selected from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
wherein:
R⁴⁴ is selected from any naturally occurring amino acid sidechain;
R⁴⁵ is selected from H, methyl, and (C₁-C₄)alkoxycarbonyl;
R⁴⁰ and R⁴¹ are selected independently from hydrogen and (C₁-C₆)hydrocarbyl;
R⁴² is (C₁-C₅)alkyl;
R⁴³ is (C₁-C₃)alkyl;
R⁵⁰ is H or (C₁-C₃)alkyl; and
R², R³, R⁴ and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
with the proviso that Ar includes 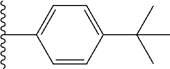 [SMILES: *c1ccc(C(C)(C)C)cc1]
only when:
W⁹ is C;
W¹ is N—R¹; and
R¹ is not H or (C₁-C₃)alkyl.

21. The method of claim 20, wherein:
W² and W⁵ are C—R² and C—R⁵, respectively; and
R², R³, R⁴, and R⁵ are independently selected from H, deuterium, halo, (C₁-C₃)alkoxy, perfluoro(C₁-C₃)alkoxy, nitrile, amino, hydroxyl, aminocarbonyl, (C₁-C₃)alkyl, and perfluoro(C₁-C₃)alkyl.

22. The method of claim 20, wherein:
W¹ is N—H or C—H;
W² and W⁵ are independently selected from C—H, C—F, C-D, C—CF₃, C—CH₃, C—Cl, C—Br, C—OH, C—OCH₃, C—NH₂, C—CF₂H, C—OCF₃, C—OCF₂H, C-CD₃, and C—CONH₂;
W³ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br; and
W⁴ is selected from N, C—H, C—NH₂, C—F, C—CF₃, C-D, C—OCH₃, C—CN, C—OH, C—Cl, C—CH₃, C—CF₂H, C—OCF₃, C—OCF₂H, C-CD₃, and C—Br.

23. The method of claim 20, wherein:
W² and W⁵ are independently selected from C—H, C—F, C-D, C—CF₂H, C-CD₃, and C—CF₃;
W³ is selected from N, C—H, C—NH₂, C—F, C—CF₃, C—CF₂H, C-CD₃, and C-D; and
W⁴ is selected from N, C—H, C—NH₂, C—F, C—CF₃, C—CF₂H, C-CD₃, and C-D.

24. The method of claim 20, wherein:
from one to four of W², W³, W⁴ and W⁵ are C-D or C—F.

25. The method of claim 20, wherein:
one of R², R³, R⁴ and R⁵ is perfluoro(C₁-C₄)alkyl, (C₁-C₄)alkyl, (C₁-C₄)alkoxy, perfluoro(C₁-C₄)alkoxy, amino, hydroxy, nitrile, halo or carboxamide, and the remainder are hydrogen.

26. The method of claim 20, wherein:
one of W³ or W⁴ is N and the other, along with W² and W⁵, is C—H.

27. The method of claim 20, wherein:
W², W³, W⁴, and W⁵ are C—H.

28. The method of claim 1, wherein the compound is selected from:
2-(4-(tert-Butyl)phenyl)-1H-imidazo[4,5-c]pyridine;
2-(4-tert-Butylphenyl)-1,3-benzothiazole;
2-(4-tert-Butylphenyl)-1,3-benzoxazole;
2-(4-tert-Butylphenyl)imidazo[1,2-a]pyridine;
2-(2-Chlorophenyl)-1H-benzo[d]imidazole;
2-(4-Chlorophenyl)-1H-benzo[d]imidazole;
2-(3-Methylphenyl)-1H-benzo[d]imidazole;
2-(6-tert-Butyl-3-pyridyl)-1H-benzo[d]imidazole;
2-(4-Methylphenyl)-1H-benzo[d]imidazole;
2-(2-Methylphenyl)-1H-benzo[d]imidazole;
2-(6-tert-Butyl-3-pyridyl)-1H-imidazo[4,5-c]-pyridine;
2-(Phenyl)-1H-benzo[d]imidazole;
2-(4-Trifluoro-methylphenyl)-1H-benzo[d]imidazole;
2-(2-Hydroxy-phenyl)-1H-benzo[d]imidazole;
2-(2-Fluorophenyl)-1H-benzo[d]imidazole;
2-(4-Cyanophenyl)-1H-benzo[d]imidazole;
2-(3-Bromophenyl)-1H-benzo[d]imidazole;
2-(3-Methoxy-phenyl)-1H-benzo[d]imidazole;
2-(4-Methoxy-phenyl)-1H-benzo[d]imidazole;
2-(2-Trifluoro-methylphenyl)-1H-benzo[d]imidazole;
2-(2-Bromophenyl)-1H-benzo[d]imidazole;
2-(4-Hydroxy-phenyl)-1H-benzo[d]imidazole;
2-(2-Methoxy-phenyl)-1H-benzo[d]imidazole;
2-(4-Bromophenyl)-1H-benzo[d]imidazole;
2-(3-Chlorophenyl)-1H-benzo[d]imidazole;
2-(4-Ethylphenyl)-1H-benzo[d]imidazole;
2-(4-tert-Butylphenyl)-5-methoxy-1H-benzo[d]imidazole;
2-(3-Cyanophenyl)-1H-benzo[d]imidazole;
2-(4-tert-Butylphenyl)-1H-benzo[d]imidazole-5-carbonitrile;
2-(4-tert-Butylphenyl)-1H-benzo[d]imidazol-5-ol;
2-(4-Isopropylphenyl)-1H-benzo[d]imidazole;
2-(4-Fluorophenyl)-1H-benzo[d]imidazole;
2-(4-tert-Butylphenyl)-1H-imidazo[4,5-b]pyridine;
2-(3-Fluorophenyl)-1H-benzo[d]imidazole;
2-(4-tert-Butylphenyl)-5-chloro-1H-benzo[d]imidazole;
2-(4-tert-Butylphenyl)-5-methyl-1H-benzo[d]imidazole;
2-(3-Trifluoromethylphenyl)-1H-benzo[d]imidazole;
2-(3-Hydroxyphenyl)-1H-benzo[d]imidazole;
2-(4-tert-Butylphenyl)-5-fluoro-1H-benzo[d]imidazole; 2-(4-tert-Butylphenyl)-5-(trifluoromethyl)-1H-benzo[d]imidazole;
2-(4-tert-Butylphenyl)-5-bromo-1H-benzo[d]imidazole;
2-(4-tert-Butylphenyl)-1H-benzo[d]imidazol-5-amine;
2-(4-tert-Butylphenyl)-4-methyl-1H-benzo[d]imidazole;
2-(4-tert-Butylphenyl)-4-chloro-1H-benzo[d]imidazole;
2-(3-Aminophenyl)-1H-benzo[d]imidazole;
2-(2-Aminophenyl)-1H-benzo[d]imidazole;
2-(4-tert-Butylphenyl)-4-fluoro-1H-benzo[d]imidazole;
2-(4-tert-Butylphenyl)-1H-benzo[d]imidazol-4-ol;
2-(4-tert-Butylphenyl)-4-methoxy-1H-benzo[d]imidazole;
2-(4-tert-Butylphenyl)-5,6-difluoro-1H-benzo[d]imidazole;
2-(3,5-di-tert-Butylphenyl)-1H-benzo[d]imidazole;
2-(4-tert-Butylphenyl)-4-bromo-1H-benzo[d]imidazole;
2-(4-tert-Butylphenyl)-4,5-difluoro-1H-benzo[d]imidazole;
2-(4-tert-Butylphenyl)-4-(trifluoromethyl)-1H-benzo[d]imidazole;
2-(4-tert-Butylphenyl)-4,6-difluoro-1H-benzo[d]imidazole;
2-(3-tert-Butylphenyl)-1H-benzo[d]imidazole;
2-[4-[1-(Trifluoromethyl)-cyclopropyl]phenyl]-1H-benzo[d]imidazole;
2-(4-tert-Butylphenyl)-4,5,6,7-tetradeuterio-1H-benzo[d]imidazole;
2-(4-tert-Butylphenyl)-4,6-dideuterio-1H-benzo[d]imidazole;
2-(4-tert-Butylphenyl)-1H-benzo[d]imidazol-4-amine;
2-(4-tert-Butylphenyl)-4,5,6,7-tetrafluoro-1H-benzo[d]imidazole;
2-(4-tert-Butylphenyl)-4,5,6-trifluoro-1H-benzo[d]imidazole;
2-(4-Biphenyl)-1H-benzo[d]imidazole;
2-(4-Trifluoromethoxyphenyl)-1H-benzo[d]imidazole;
2-(4-Ethoxyphenyl)-1H-benzo[d]imidazole;
2-(4-Propoxyphenyl)-1H-benzo[d]imidazole;
2-(4-Isopropoxyphenyl)-1H-benzo[d]imidazole;
2-(4-(Difluoromethoxy)phenyl)-1H-benzo[d]imidazole;
2-(4-tert-Butoxyphenyl)-1H-benzo[d]imidazole;
2-(4-Phenoxyphenyl)-1H-benzo[d]imidazole;
2-(4-Benzyloxyphenyl)-1H-benzo[d]imidazole;
2-(4-tert-Butylphenyl)-1H-benzo[d]imidazol-4-carboxamide;
2-(4-tert-Butylphenyl)-1H-benzo[d]imidazol-5-carboxamide;
2-(4-Aminophenyl)-1H-benzo[d]imidazole;
2-(3-tert-Butylphenyl)-4,6-difluoro-1H-benzo[d]imidazole;
4,6-Difluoro-2-(4-methoxyphenyl)-1H-benzo[d]imidazole;
2-(4-(tert-Butyl)phenyl)-1H-benzo[d]imidazole-4-carboxylic acid;
2-(4-(tert-Butyl)phenyl)-1H-benzo[d]imidazole-5-carboxylic acid;
2-(4-(tert-Butyl)phenyl)imidazo[1,2-a]pyrimidine; and
4-(4,6-Difluoro-1H-benzo[d]imidazol-2-yl)thiazole.

29. The method of claim 1, wherein the compound is 2-(4-tert-butylphenyl)-1H-benzo[d]imidazol-5-ol having the formula:

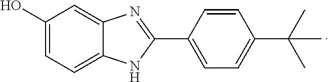 [SMILES: CC(C)(C)c1ccc(-c2nc3cc(O)ccc3[nH]2)cc1]

30. The method of claim 1, further comprising administering to the subject one or more additional active agents.

31. The method of claim 30, wherein the one or more additional active agents is selected from hydroxyurea, L-glutamine, crizanlizumab and voxelotor, or pharmacutically acceptable salts thereof.

32. The method of claim 31, further comprising administering to the subject hydroxyurea.

33. The method of claim 32, wherein the subject is a human.

34. The method of claim 33, wherein the human is no more than 40 years of age.

35-38. (canceled)

39. A method of increasing fetal hemoglobin (HbF) production, the method comprising administering to the subject in need of increased HbF production an effective amount of a compound of formula I: 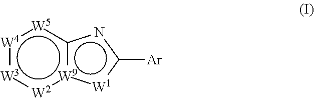
or a pharmaceutically acceptable salt, solvate, deuterated derivative, or prodrug thereof,
wherein:
Ar is 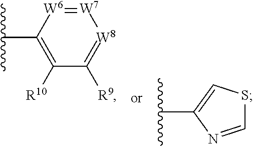
W¹ is chosen from O, S, and N—R¹, or, when W⁹ is N, W¹ may additionally be C—R⁵⁰;
W² is N or C—R²;
W³ is N or C—R³;
W⁴ is N or C—R⁴;
W⁵ is N or C—R⁵;
W⁶ is N or C—R⁶;
W⁷ is N or C—R⁷;
W⁸ is N or C—R⁸;
W⁹ is C, or, when W¹ is C—R⁵⁰; W⁹ may be N;
wherein:
R¹ is chosen from H, (C₁-C₃)alkyl, —CH₂OC(═O)R³⁰; —CH₂OP(═O)OR⁴⁰OR⁴¹, —C(═O)OR⁴², and —C(═O)R⁴³;
wherein:
R³⁰ is chosen from (C₁-C₁₀)hydrocarbyl, (C₁-C₁₀)hydrocarbyl substituted with amino, (C₁-C₁₀)hydrocarbyl substituted with (C₁-C₄)alkoxycarbonyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵, and guanidino;
wherein:
R⁴⁴ is chosen from any naturally occurring amino acid sidechain;
R⁴⁵ is chosen from H, methyl, and (C₁-C₄)alkoxycarbonyl;
R⁴⁰ and R⁴¹ are chosen independently from hydrogen and (C₁-C₆)hydrocarbyl;
R⁴² is (C₁-C₅)alkyl;
R⁴³ is (C₁-C₃)alkyl; and
R⁵⁰ is H or (C₁-C₃)alkyl;
R², R³, R⁴ and R⁵ are chosen independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino[—HNC(═O)O-alkyl], carboxamido[—C(═O)NH₂], (C₁-C₄)alkylaminocarbonyl [—C(═O)NH-alkyl], cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are chosen independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are chosen independently from hydrogen, deuterium, hydroxy, cyano, amino, halogen, halo(C₁-C₄)alkyl, (C₁-C₄)alkyl, (C₁-C₄)alkoxy, and halo(C₁-C₄)alkoxy, 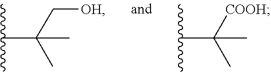
R⁸ is chosen from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, and amino, 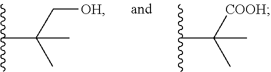
with the proviso that:
i) when Ar is 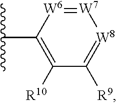
R⁸ is not (C₁-C₄)alkyl when:
(a) W¹ is N—R¹;
(b) R¹ is hydrogen;
(c) W², W³, W⁴, W⁵, W⁶, and W⁷ are C—H;
(d) W¹ is C—R⁸;
(e) W⁹ is C; and
(f) R⁹ and R¹⁰ are hydrogen.

40. A method of treating a hemoglobinopathy, comprising:
administering to a subject suffering from a hemoglobinopathy an effective amount of a pharmaceutical composition comprising an amorphous solid dispersion of 2-(4-tert-butylphenyl)-1H-benzimidazole (TQS-168) or a pharmaceutically acceptable salt, solvate, deuterated derivative, or prodrug thereof; and a pharmaceutically acceptable carrier matrix.

41-111. (canceled)

112. A method of increasing fetal hemoglobin (HbF) production, the method comprising:
administering to a subject in need of increased HbF production an effective amount of a pharmaceutical composition comprising an amorphous solid dispersion of 2-(4-tert-butylphenyl)-1H-benzimidazole (TQS-168) or a pharmaceutically acceptable salt, solvate, deuterated derivative or prodrug thereof, and
a pharmaceutically acceptable carrier matrix.
